(19) United States
(12) Patent Application Publication
(10) Pub. No.: US 2017/0183653 A1
(43) Pub. Date: Jun. 29, 2017

(54) Pri-mirna libraries and methods for making and using pri-mirna libraries

(71) Applicant: Alan Marc Hoskins Beem, Seattle, WA (US)

(72) Inventor: Alan Marc Hoskins Beem, Seattle, WA (US)

(21) Appl. No.: 15/313,000

(22) PCT Filed: May 23, 2015
(86) PCT No.: PCT/US15/00062

§ 371 Date: Nov. 21, 2016

(51) Int. Cl. C12N 15/113; C12N 15/10

(52) U.S. Cl. CPC C12N 15/113; C12N 15/1079; C12N 2310/141; C12N 2330/31

(57) ABSTRACT

Provided are methods for the design, preparation, and use of non-native primicroRNAs (pri-miRNAs), pri-microRNA (pri-miRNA) scaffolds, and libraries of non-native pri-miRNAs employing pri-miRNA scaffolds. Also provided are methods for identifying nonnative pri-miRNAs, combinations of two or more non-native pri-miRNAs, and miRNAs derived from the processing of such non-native pri-miRNAs, which miRNAs exhibit one or more desired functional activities. Further provided are non-native pri-miRNAs, non-native pri-miRNA libraries, vectors comprising and for the expression of one or more non-native pre-miRNAs or for the expression of one or more miRNAs derived from the processing of one or more premiRNAs, and cells comprising one or more non-native pri-miRNAs or one or more miRNAs derived from the processing of such non-native pri-miRNAs, each of which pri-miRNAs, primiRNA libraries, vectors, and cells can be prepared by the methods disclosed herein.

### CROSS-REFERENCE TO RELATED APPLICATIONS

This application claims the benefit of U.S. Provisional Patent Application No. 62/002,069, which was filed on May 22, 2014. U.S. Provisional Patent Application No. 62/002,069 is incorporated herein by reference in its entirety.

### BACKGROUND OF THE DISCLOSURE

Technical Field

The present disclosure relates, generally, to RNA interference, in particular to non-native pri-microRNAs (pri-miRNAs) and libraries comprising non-native pri-miRNAs. More specifically, this disclosure provides: (1) methods for the design, preparation, and use of pri-miRNA scaffolds that may be employed for use in the preparation of pri-miRNA libraries; (2) methods for the design, preparation, and use of pri-miRNA libraries employing those pri-miRNA scaffolds; (3) methods for identifying individual non-native pri-miRNAs and combinations of two or more non-native pre-miRNAs that, when processed in vivo, yield one or more miRNAs exhibiting one or more desired functional activities; (4) pri-miRNAs, libraries of pri-miRNAs, vectors systems for the intracellular expression of pri-miRNAs, and cells comprising one or more pri-miRNAs or one or more vectors for the expression of one or more pri-miRNAs; (5) methods for the regulation, promotion, normalization, restoration, inhibition, or modulation of one or more cellular phenotype (e.g., cell differentiation, cell proliferation, cell growth, cell death, cell-cell contact inhibition), which methods employ one or more pri-miRNAs, such as pri-miRNAs identified through the screening of a pri-miRNA library according to the methodology disclosed herein; and (6) methods for the treatment of a disease or condition that is associated with the expression of one or more gene or the production of one or more protein, wherein one or more aspect of the disease or condition is reduced in severity following the expression of one or more pri-miRNAs or one or more miRNAs identified through the screening of a pri-miRNA library, or otherwise prepared, according to the methodology disclosed herein.

Description of the Related Art

MicroRNAs (miRNAs) are small RNA molecules that direct translational repression. The biosynthesis of 19-23 nucleotide miRNAs is initiated within the nucleus of eukaryotic cells with the cleavage of primary-miRNA (pri-miRNA) transcripts (>200 nt) by the RNase III enzyme Drosha and its cofactor DGCR8. Upon transport to the cytoplasm, the RNase III enzyme Dicer performs an additional processing step.

More specifically, Drosha removes the single-stranded flanks that are characteristic of pri-miRNAs and 11 nucleotides of the stem to form pre-miRNAs. Pri-miRNAs are exported from the nucleus by Exportin5, which is GTP-dependent. In the cytoplasm, Dicer removes the terminal loop from pri-miRNAs, forming a 19-23 nt RNA duplex called “miR:miR*”, representing the guide strand and passenger strand, respectively. miRs are preferentially loaded into the RNA-induced silencing complex (RISC), formed by TRBP, Ago2, and Dicer. In association with RISC, the miRs guide the complex to bind mRNAs with target sequences that imperfectly base-pair with an miR ‘seed region’ of at nucleotides 2-7 from the 5′-end of the miR.

Target sequences for miRNA are found within mRNA 3′- and 5′ untranslated regions (UTRs) as well as within mRNA coding regions. Some miRNAs target single mRNAs at multiple sites. MiRNA seeds are predicted to target on the order of 200 genes each, and most mRNA are targeted by multiple miRNA. Upon binding of RISC-associated miRs, the target mRNAs become subject to translational arrest, mRNA degradation, or mRNA de-adenylation. In addition to the regulatory effects of miRNA, paradigms of post-transcriptional regulation of pri-miRNA-maturation are emerging from current studies that reveal the tight regulation of miRNA maturation.

Modern paradigms for the regulation of pri-miRNA-maturation depend upon either the sequence-specific or the sequence independent binding of proteins to pri-miRNAs. Some post-transcriptional regulation serves to integrate extracellular signals. For example, when stimulated by BMP or TGF-β signaling, SMAD1, 3, and 5 interact with p68 and Drosha to promote the processing of miR-21. Some regulation of pri-miRNA maturation depends upon binding between the terminal loop of the pri-miRNA and proteins such as KSRP, or hnRNPA1, which promote processing by both Drosha and Dicer. Some interactions also inhibit miRNA processing, such as the interaction of E2-activated ERa with p68 to inhibit Drosha processing, or LIN28B, which inhibits processing of the let-7 family by both Drosha and Dicer.

Regulation of the miRNA biosynthesis pathway constrains the possible miRNA profiles that result from a given set of DNA sequences, RNA molecules, transcription factors, and other proteins present in a cell. In turn, miRNA profiles constrain mRNA and protein expression profiles. Through the combined effects of pri-miRNA expression and integration of cellular signals, the miRNA pathway exerts global and context specific control over gene expression in the cell.

Recent advances in the use of miRNAs to control cell fate, and the necessity of miRNAs for many cellular functions, suggest the use of miRNAs to control cell fate or to induce or aid in the differentiation of stem cells, such as embryonic stem cells (ESCs) and induced pluripotent stem cells (iPSCs) into specific cell types. Moreover, particular miRNA expression profiles are associated with many cellular processes beyond stem cell maintenance, proliferation, and differentiation. The use of ectopic miRNA expression to aid in cellular differentiation or to induce stem-ness has been reported. For example, Shenoy and Blelloch observed that the expression of miR-124 and miR-9 is sufficient to induce transdifferentiation of fibroblasts into neuron-like cells. Shenoy and Blelloch, F1000 Biology Reports 4:3 (2012).

In vitro cell cultures are indispensable tools for the study of developmental neurobiology, functioning neural networks, mental health diseases, and neuropharmacology. In vivo research is also necessary, but culturing cells outside the body allows the quantification and control of molecular and electrical characteristics of cells to a degree that is not possible in most organisms, especially humans. In vitro experiments have become increasingly useful with advances in the ability of investigators to control and quantify cellular characteristics, most notably the advent of techniques to culture hESC or iPSC and direct their differentiation into specific cell types.

Notwithstanding recent advances in the understanding of miRNA-mediated regulation of cellular phenotypes, there remains an unmet need in the art for technologies that permit the rapid identification of pri-miRNAs that, either individually or in combination, are processed into miRNAs that can effect one or more cellular functions thereby regulating, promoting, normalizing, restoring, inhibiting, or modulating one or more cellular phenotype such as, for example, cell differentiation, proliferation, growth, death, or contact inhibition.

### SUMMARY OF THE DISCLOSURE

The present disclosure fulfills certain unmet needs in the art by providing non-native pri-microRNAs (pri-miRNAs), libraries comprising those pri-miRNAs, pri-miRNA scaffolds for generating libraries comprising those pri-miRNAs, methods for making and using those miRNA scaffolds, non-native pri-miRNAs, and libraries of non-native pri-miRNAs. More specifically, the presently disclosed pri-miRNA libraries may be advantageously employed for the identification of pri-miRNAs that can regulate, promote, normalize, restore, inhibit, or modulate one or more cellular function (such as, for example, cell maintenance, survival, growth, proliferation, differentiation, or death) or that can regulate, promote, normalize, restore, inhibit, or modulate one or more cellular phenotype. Such non-native pri-miRNAs as those disclosed herein may be used in methods for reducing in severity one or more aspect of a disease or condition that is associated with one or more cellular function or cellular phenotype, in particular a cellular function or phenotype that is caused by or associated with the altered (e.g., elevated or reduced) expression of one or more genes or the altered (e.g., elevated or reduced) production of one or more proteins.

Within certain embodiments, the present disclosure provides pri-miRNAs and miRNAs resulting from the processing of those pri-miRNAs, libraries of pri-miRNAs, vectors for the expression of pri-miRNAs and miRNAs derived from the processing of such pri-miRNAs, and cells comprising such pri-miRNA or miRNA expression vectors.

Within certain aspects of these embodiments, pri-miRNA scaffolds can include, in various combinations as described in detail herein: (1) one or more palindromic sequences for facilitating pri-miRNA hairpin formation; (2) one or more Drosher/DGCR8 binding sequences; and (3) one or more sites for the insertion of one or more miRNA sequences or one or more miRNA seed sequences.

Within other aspects of these embodiments, pri-miRNA libraries can include, in various combinations as described in detail herein: (1) one or more palindromic sequences for facilitating pri-miRNA hairpin formation; (2) one or more Drosher/DGCR8 binding sequences; and (3) one or more miRNA sequences comprising one or more miRNA seed sequences and one or more flanking sequences, wherein the miRNA seed sequences or the flanking sequences are either fully-randomized or partially-randomized such that the pri-miRNA libraries have a complexity of pri-miRNAs having unique seed sequences or flanking sequences of from 10⁴ distinct pri-miRNAs to 10⁹ distinct pri-miRNAs, or from 10⁵ distinct pri-miRNAs to 10⁸ distinct pri-miRNAs, or from 10⁶ distinct pri-miRNAs to 10⁷ distinct pri-miRNAs.

Within other embodiments, the present disclosure provides methods for the design, preparation, and use of pri-miRNA scaffolds; non-native pri-miRNAs employing those pri-miRNA scaffolds; and pri-miRNA libraries of those non-native pri-miRNAs. Within certain aspects of these embodiments, methods for the use of pri-miRNA libraries include the unbiased selection of one or more pri-miRNAs that can effect a phenotypic change upon a target cell, such as stem cell, a partially or terminally differentiated cell, or a cell that is associated with a disease or other condition. Within related aspects of these embodiments, screening of the pri-miRNA libraries is performed without regard to any specific target sequence or other known or presumed factor that is associated with the phenotypic change, differentiation state, disease, or condition. In such a manner, the presently disclosed methods for use of pri-miRNA libraries for selecting one or more pri-miRNAs permit the identification of pri-miRNAs having specificity for one or more intracellular not previously known to be associated with the phenotypic change, differentiation state, disease, or condition.

Within other embodiments, the present disclosure provides methods for the design, preparation, and use of pri-miRNA libraries of non-native pri-miRNAs comprising one or more pri-miRNA scaffolds as disclosed herein.

Within other embodiments, the present disclosure provides methods for identifying pri-miRNAs and combinations of two or more pri-miRNAs that, when processed intracellulary, yield miRNAs exhibiting one or more desired functional activities.

Within other embodiments, the present disclosure provides methods for regulating, promoting, normalizing, restoring, inhibiting, or modulating a desired cellular phenotype including, for example, differentiation, de-differentiation, proliferation, growth, cell death, contact inhibition by expressing one or more miRNAs identified through the screening of a miRNA library according to the methodology disclosed herein.

Within other embodiments, the present disclosure provides methods for the treatment of a disease or condition that associated with the expression of one or more gene or the production of one or more protein, wherein one or more aspect of the disease or condition is reduced in severity following the expression of one or more miRNAs identified through the screening of a miRNA library according to the methodology disclosed herein.

It will be understood that various changes, alterations, and substitutions may be made to the specific embodiments disclosed herein without departing from their essential spirit and scope. Certain aspects of these embodiments will become more evident to those possessing skill in the art when reference is made to the following detailed description in conjunction with the accompanying drawings.

### BRIEF DESCRIPTION OF THE DRAWINGS

FIG. 1 is a diagram depicting that major events in the in vivo biogenesis of a mature miRNA from a corresponding pri-miRNA.

FIG. 2 is a diagram depicting the construction of an exemplary pri-miRNA library according to the methods disclosed herein.

FIG. 3 is a diagram depicting the construction of an exemplary pri-miRNA scaffold according to the methods disclosed herein.

FIG. 4 is a diagram of an exemplary miRNA (i.e., human miR-1) showing the palindromic sequence, which defines the miRNA's ultimate hairpin structure.

FIGS. 5A and 5B are alignments of nucleotide sequences for a collection of representative pri-miRNAs. Highlighted nucleotides indicate regions of sequence conservation within the palindromic sequences.

FIG. 6 is a diagram of a consensus pri-miRNA structure derived through RNAalifold analysis of the pri-miRNA nucleotide sequences presented in FIGS. 5A and 5B.

### DETAILED DESCRIPTION OF THE DISCLOSURE

The present disclosure is based upon the development of pri-miRNA scaffolds, non-native pri-miRNAs comprising a pri-miRNA scaffold and one or more polynucleotide sequences comprising a miRNA seed sequence and flanking regions, libraries of non-native pri-miRNAs comprising a pri-miRNA scaffold and one or more polynucleotide sequences comprising a miRNA seed sequence and flanking regions, and methods for the design, preparation, and use of those pri-miRNA scaffolds, non-native pri-miRNAs, and libraries of non-native pri-miRNAs.

As discussed in further detail herein, the presently disclosed pri-miRNA libraries may be advantageously employed in an unbiased fashion to efficiently identify one or more non-native pri-miRNAs that exhibit one or more desired functionality such as, for example, the regulation, promotion, normalization, restoration, inhibition, or modulation of one or more cellular function (such as, for example, cell maintenance, survival, growth, proliferation, differentiation, or death) or that can regulate, promote, normalize, restore, inhibit, or modulate one or more cellular phenotype.

Within certain embodiments, the non-native pri-miRNAs identified through the screening of the presently disclosed pri-miRNA libraries may be used in methods for reducing in severity one or more aspect of a disease or condition that is associated with one or more cellular function or cellular phenotype, in particular a cellular function or phenotype that is caused by or associated with the altered (e.g., elevated or reduced) expression of one or more genes or the altered (e.g., elevated or reduced) production of one or more proteins.

Within other embodiments, the non-native pri-miRNAs identified through the screening of the presently disclosed pri-miRNA libraries may be used in methods for promoting the differentiation of an undifferentiated cell, such as a stem cell, to a partially or fully committed cell of a desired cell lineage. Conversely, the non-native pri-miRNAs identified through the screening of the presently disclosed pri-miRNA libraries may be used in methods for promoting the de-differentiation of a differentiated cell, such as a fibroblast or other partially or fully committed cell of a particular cell lineage to an undifferentiated cell, such as a stem cell, in particular an induced pluripotent stem cell (iPSC).

Based, in part, on the importance of pri-miRNA expression, and the intracellular processing of pri-miRNAs into mature miRNAs, in determining cellular state and integrating cellular signals as well as on the essential function of a seed sequences and sequences flanking those seed sequences in conferring mRNA target specificity, the present disclosure provides libraries of non-native pri-miRNAs that may be used in the methods disclosed herein for facilitating the rapid, unbiased screening for and identification of non-native pri-miRNAs that, when processed into mature miRNAs, can, either alone or in combination, effect one or more desired cellular functions, cellular activities, or cellular phenotypes.

Thus, the present disclosure provides pri-miRNA scaffolds, non-native pri-miRNAs generated with and comprising a pri-miRNA scaffold, and libraries of such non-native pri-miRNAs that comprise non-native pri-miRNAs generated with and comprising a pri-miRNA scaffold as disclosed herein. Also provided are methods for making and using such non-native pri-miRNAs, pri-miRNA scaffolds, and non-native pri-miRNA libraries. Pri-miRNAs, pri-miRNA scaffolds, and pri-miRNA libraries that are made by the methods provided herein will find broad application for the intracellular delivery of pri-miRNAs and for the intracellular processing of pri-miRNAs into biologically active, yet non-native miRNAs that may be used for regulating, modulating, normalizing, and/or regulating one or more cellular functions, cellular activities, or cellular phenotypes.

The non-native pri-miRNAs disclosed herein employ a pri-miRNA scaffold that comprises one or more of the following features: (1) one or more palindromic sequences for facilitating pri-miRNA hairpin formation; (2) one or more Drosher/DGC8 binding sequences; and (3) one or more sites for the insertion of one or more miRNA sequences or one or more miRNA seed sequences.

The presently disclosed pri-miRNA scaffolds and pri-miRNAs comprising those pri-miRNA scaffolds are designed for the production of pri-miRNA libraries having a complexity of at least 10⁵ distinct miRNAs. For example, the miRNA libraries disclosed herein have a complexity of from 10⁵ distinct pri-miRNAs to 10⁹ distinct pri-miRNAs or from 10⁶ distinct pri-miRNAs to 10⁸ distinct pri-miRNAs.

Such pri-miRNA libraries may generated by the methods disclosed herein and may be advantageously employed in methods for regulating, promoting, normalizing, restoring, inhibiting, or modulating a desired cellular phenotype (e.g., cell maintenance, survival, growth/proliferation, differentiation, or death) and in methods for reducing in severity one or more aspect of a disease or condition that is associated with such a cellular phenotype, in particular a cellular phenotype that is associated with an altered (e.g., elevated or reduced) expression of one or more gene or an altered (e.g., elevated or reduced) production of one or more protein.

Within other embodiments, the present disclosure provides methods for making pri-miRNAs scaffolds and pri-miRNAs comprising a pri-miRNA scaffold. Within other embodiments, the present disclosure provides methods for making pri-miRNA libraries comprising pri-miRNAs each of which comprises a pri-miRNA scaffold and a 19-23 nucleotide sequence containing one or more seed sequences as disclosed herein. Within other embodiments, the present disclosure provides methods for using the pri-miRNA libraries disclosed herein for the identification of individual pri-miRNAs and groups of pri-miRNAs that regulate, promote, normalize, restore, inhibit, or modulate a desired cellular phenotype (for example, cell maintenance, survival, growth/proliferation, differentiation, or death) and reduce in severity one or more aspect of a disease or condition that is associated with such a cellular phenotype, in particular a cellular phenotype that is associated with an altered (e.g., elevated or reduced) expression of one or more gene or an altered (e.g., elevated or reduced) production of one or more protein.

The practice of the present disclosure will employ, unless indicated specifically to the contrary, conventional methodology and techniques that are in common use in the fields of virology, oncology, immunology, microbiology, molecular biology, and recombinant DNA, which methodology and techniques are well known by and readily available to those having skill of the art. Such methodology and techniques are explained fully in laboratory manuals as well as the scientific and patent literature. See, e.g., Sambrook, et al., “Molecular Cloning: A Laboratory Manual” (2nd Edition, Cold Spring Harbor Laboratory Press, Cold Spring Harbor, N.Y., 1989); Maniatis et al., “Molecular Cloning: A Laboratory Manual” (1982); “DNA Cloning: A Practical Approach, vol. I & II” (Glover, ed.); “Oligonucleotide Synthesis” (Gait, ed., 1984); Ausubel et al. (eds.), “Current Protocols in Molecular Biology” (John Wiley & Sons, 1994); “Nucleic Acid Hybridization” (Hames & Higgins, eds., 1985); “Transcription and Translation” (Hames & Higgins, eds., 1984); “Animal Cell Culture” (Freshney, ed., 1986); and Perbal, “A Practical Guide to Molecular Cloning” (1984). All publications, patents and patent applications cited herein, whether supra or infra, are hereby incorporated by reference in their entirety.

### Non-Native Pri-microRNAs, Pri-microRNA Scaffolds, and Methods for their Design and Preparation

Within certain embodiments, the present disclosure provides non-native pri-mRNAs, pri-microRNA scaffolds, and methods for the design and preparation of such pri-miRNA scaffolds, which pri-miRNA scaffolds employ, in various combination: (1) one or more palindromic sequences for facilitating miRNA hairpin formation; (2) one or more Drosher/DGCR8 binding sequences; and (3) one or more sites for the insertion of one or more miRNA sequences, which miRNA sequences contain one or more miRNA seed sequences.

As described herein, certain pri-miRNA scaffolds of the present disclosure can be employed, generally, in methods for making non-native pri-miRNA and libraries of pri-miRNA, which pri-miRNA libraries can be advantageously employed for the identification of one or more pri-miRNA having a desired intracellular activity such as, for example, regulating, modulating, normalizing, and/or restoring one or more cellular functions, such as one or more of cell maintenance, survival, growth, proliferation, differentiation, and/or death.

Mature miRNAs are small non-coding RNA molecules having lengths of from about 19 nucleotides and 23 nucleotides that function in RNA silencing and post-transcriptional regulation of gene expression. MiRNAs function via base-pairing with complementary sequences within a target mRNA, which leads to mRNA silencing by one or more following processes, including: (1) target mRNA cleavage, (2) destabilization of a target mRNA by truncation of its polyA tail, and (3) reducing the efficiency of target mRNA translation. It is estimated that the human genome encodes over 1000 miRNAs and target about 60% of human genes.

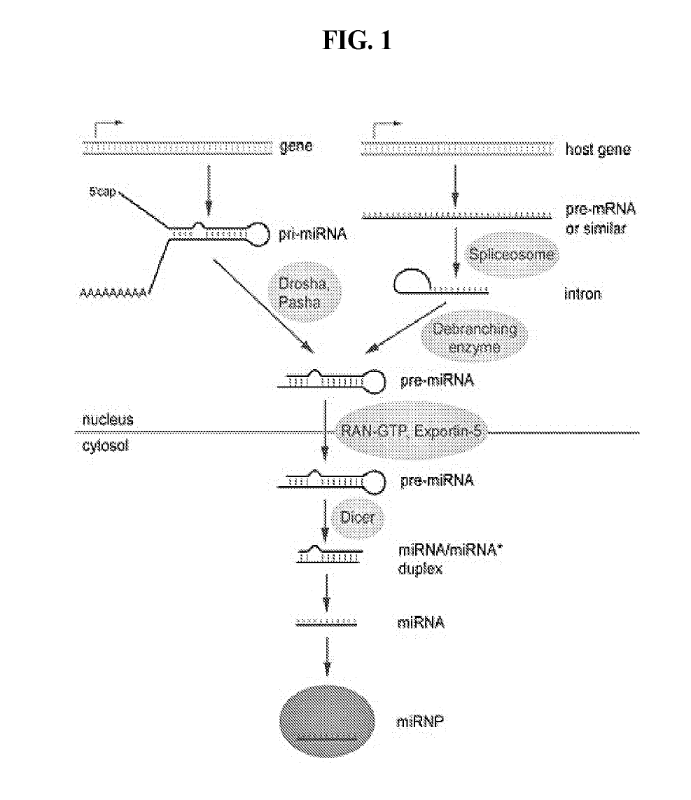

MiRNAs resemble small interfering RNAs (siRNAs) of the RNA interference (RNAi) pathway. MiRNAs differ in origin from siRNAs, however. That is, while siRNAs derive from longer regions of double-stranded DNA, miRNAs derive from pri-miRNAs characterized by the formation of short hairpins. See, FIG. 1, which presents a diagrammatic representation of miRNA biogenesis.

Human miRNAs specifically bind to target mRNAs through a seed region of 6-8 nucleotides at the 5′ end of the miRNA. The seed region, alone, is, however, insufficient for inducing cleavage of the target mRNAs. Recent estimates predict that a single miRNAs has, on average, roughly 400 conserved targets and can reduce the stability of hundreds of unique messenger RNAs anc can repress the production of hundreds of proteins.

Genes encoding pri-miRNAs are usually transcribed by RNA polymerase II (Pol II) or RNA polymerase III (Pol III), which binds to a promoter near a DNA sequence encoding a pri-miRNA hairpin loop. Pri-miRNA transcripts are capped at their 5′ end, polyadenylated, and spliced. Pri-miRNAs are transcribed as part of one arm of an ˜80 nucleotide RNA stem-loop that forms part of a several hundred nucleotide long pri-miRNA.

A single pri-miRNA may contain from one to six miRNA precursors. These hairpin loop structures of about 70 nucleotides in length. Each hairpin is flanked by sequences tat are required for efficient processing. The double-stranded RNA structure of hairpins in a pri-miRNA is recognized by the nuclear protein DiGeorge Syndrome Critical Region 8 (DGCR8). DGCR8 associates with the pri-miRNA processing enzyme Drosha, which binds the pri-miRNA to form the “Microprocessor” complex. DGCR8 orients the catalytic RNase III domain of Drosha, which cleave the pri-miRNA at about 11 nucleotides distal to the hairpin base (i.e., about two helical RNA turns into the stem) thereby excising hairpins from pri-miRNAs. The resulting digestion product, which is referred to as a precursor-miRNA (pre-miRNA) has has 3′ hydroxyl and 5′ phosphate groups and a two-nucleotide overhang at its 3′ end.

Pre-miRNA hairpins are exported out of the nucleus in a process mediated by the nucleocytoplasmic protein Exportin-5, which recognizes the two-nucleotide 3′ overhang that results from Drosha cleavage of the pri-miRNA. Exportin-5-mediated transport to the cytoplasm is energy-dependent, and is facilitated by the Ran protein, which uses GTP as its energy source.

In the cytoplasm, pre-miRNA hairpins are cleaved by the RNase III enzyme Dicer. This endoribonuclease interacts with the 3′ end of the hairpin and cuts away the loop joining the 3′ and 5′ arms, yielding an imperfect miRNA:miRNA* duplex of from about 19-23 nucleotides in length. Hairpin length, loop size, and imperfections in miRNA:miRNA* pairing are factors that influence the efficiency of Dicer processing and cleavage. Certain G-rich pre-miRNAs can adopt a Dicer resistant G-quadruplex structure instead of a canonical step-loop structure (e.g., human pre-miRNA 92b). Of the two strands comprising the hairpin duplex, one strand is typically incorporated into the RNA-induced silencing complex (RISC), which mediates the interaction between the miRNA and its mRNA target interact. The mature miRNA is part of an active RISC complex (a/k/a microRNA ribonucleoprotein complex, miRNP), which contains Dicer and other associated proteins.

Dicer processing of the pre-miRNA is coupled with duplex unwinding such that only one strand is incorporated into the miRNP complex. The incorporated strand is selected on the basis of its thermodynamic instability, weaker base-pairing relative to the other strand, and the position of the stem-loop. The other strand (i.e., the passenger strand) is normally degraded.

Members of the Argonaute (Ago) protein family are central to RISC function. Argonautes are needed for miRNA-induced silencing and contain two conserved RNA binding domains: a PAZ domain that can bind the single stranded 3′ end of the mature miRNA and a PIWI domain that structurally resembles ribonuclease-H and functions to interact with the 5′ end of the guide strand. They bind the mature miRNA and orient it for interaction with a target mRNA. Human Ago2 cleaves target transcripts directly while other argonautes recruit additional proteins to achieve translational repression. In humans, eight argonaute proteins comprising two families are encoded (i.e., AGO and PIWI).

Each of the structural and functional aspects of the presently-disclosed non-native pri-miRNAs, pri-miRNA scaffolds, and pri-miRNA libraries are based, in part, upon these observations regarding the molecular basis for pri-miRNA biogenesis. Those structural and functional aspects of non-native pri-miRNAs, pri-miRNA scaffolds, and pri-miRNA libraries are described in further detail herein.

MiRNAs tend to reside in short conserved segments (70% in segments of at most 200 bp) and their stems have relatively few bulges (86% have at most 20% of their bases in bulges). Rules for identifying stem-loop structures, including miRNAs, among conserved regions of multi-species genome alignments and among intergenic and intronic hairpins (stem-loops) are described in Pedersen et al., PLoS Comput. Biol. 2(4):e33 (2006).

Because compensatory substitutions in base-paired regions preserve structure, Gorodkin and colleagues have suggested that conserved secondary structure, rather than primary sequence, is the hallmark of many functionally important RNAs.” Gorodkin et al., Trends in Biotechnology 28(1):9-19 (2010).

Within certain embodiments of the present disclosure, pri-miRNA scaffolds may be generated directly from one or more wild-type pri-miRNAs. For example, a pri-miRNA scaffold of the present disclosure may be generated using a base sequence structure from naturally occurring pri-miRNAs. DGCR8/Drosha binding and known structural motifs may be used as a reference point. More specifically, it has been reported that a DGCR8/Drosha binding/cleavage site is present within a pri-miRNA hairpin structure at a position that is from 8 nucleotides to 14 nucleotides, more commonly from 11 nucleotides to 12 nucleotides, 3′ to the first base of the 5′ palindrome sequence that constitutes one strand of the pri-miRNA hairpin structure.

Exemplary pri-miRNA sequences that may be used to construct a scaffold are set forth in Table 1. These pri-miRNA sequences were palindrome optimized such that bulges and mismatches within the wild type palindrome sequence were eliminated thereby leading to the formation of idealized hairpin structures within the pri-miRNA secondary structure.

DGCR8 harbors a stem-loop structure that resembles a miRNA and that confers pri-miRNA binding capacity to the DGCR8/Drosha complex. See, Triboulet et al., RNA 15(6):1005-1011 (2009). As discussed herein, the dsRNA hairpin region is flanked by ssRNA arms of variable length and DGCR8/binding occurs within that dsRNA hairpin region. The miRNA seed sequence consists of a 2-8 nucleotide sequence beginning immediately 3′ to the DGCR8/Drosha binding site and the miR:miR* region consists of the 19-23 nucleotide sequence, which includes the 6 nucleotide seed sequence, beginning immediately 3′ to the DGCR8/Drosha binding site.

It will be understood that DGCR8/Drosha binding sites may be (1) known in the art or (2) may be identified by (a) determining a region of high self-complementarity in a given pri-miRNA candidate double-stranded DNA sequence (palindromic sequence) and (b) determining a nucleotide position that is 8-14 nucleotides, or more commonly 11-12 nucleotides, in from the 5′ end of the DGCR8/Drosha binding site. Those skilled in the art may, therefore, infer the location and sequence of both the 19-23 nucleotide miR:miR* region (i.e., 19-23 nucleotides 3′ to the DGCR8 binding site) and the seed sequence (2-8 nucleotides 3′ to the DGCR8 binding site). Alternatively, a candidate DGCR8/Drosha binding site may be determined empirically by isolating miRNA from a wild-type sample, creating a miCDNA using reverse transcriptase, sequencing the resulting miCDNA, and comparing that sequence to the sequence of a corresponding pri-miRNA.

Having thus identified the location and sequences of the various pri-miRNA regions, it will be appreciated that one or both of the miR:miR* region and the seed sequence may be manipulated, altered, or substituted using, for example, random mutagenesis, site-directed mutagenesis, RNA nucleotide deletion, or RNA nucleotide insertion to, for example, insert one or more restriction enzyme recognition sites, insert one or more RNA nucleotides, delete one or more RNA nucleotides, cross-link one or more RNA nucleotides, or alter or change the identity of one or more RNA nucleotides.

Those of skill in the art will further appreciate that novel miRNAs may be constructed through the use of such known techniques. For example, a given seed sequence may be altered to increase or otherwise modulate binding affinity for one or more known target mRNA transcripts, or may be replaced by a seed sequence associated with a target mRNA transcript. Likewise, a miR:miR* region, or a portion thereof, may be altered to increase or otherwise modulate binding affinity for one or more known target mRNA transcripts or, alternatively, may be replaced by a different miR:miR* region associated with a target mRNA transcript.

Within other aspects of the present embodiments, pri-miRNA scaffolds may be generated de novo. It may, for example, be desirable to synthesize novel pri-miRNAs without reference to a naturally-occurring pri-miRNA sequence or without reliance on a scaffold derived from a naturally-occurring pri-miRNA. Certain cell types, such as embryonic stem cells, are associated with only certain pri-miRNAs in vivo. See, e.g., Houbaviy et al., Dev. Cell. 5(2):351-8 (2003).

Novel pri-miRNAs may be advantageously employed to confer new or augmented functionalities to such cell types. In one example, an optimized pri-miRNA that is perfectly palindromic (i.e., the hairpin sequence may be engineered to eliminate any mismatches, bulges, or internal loops, such as presented in Table 1) may be introduced to a cell to improve efficiency of miRNA biosynthesis and resultant modulation of mRNA transcripts. In another example, a cell that is associated only with certain pri-miRNAs may gain a new, distinct, or improved functionality by introduction of a novel pri-miRNA. In yet another example, a cell exhibiting abnormal expression of an mRNA that is associated with a particular disease state may be treated by the introduction of a pri-miRNA that is novel to a cell of that type. It will be appreciated that a novel pri-miRNA may be constructed while preserving core structural features of pri-miRNAs, including a self-complementary palindromic dsRNA “stem” region, a hairpin loop region, and one or two ssRNA flanker regions.

Whether the pri-miRNA or pri-mirRNA scaffold is generated by reference to a naturally occurring sequence or is produced de novo, it will be appreciated that certain alterations, such as introduction of a mismatch or a wobble, or elimination of a mismatch or a wobble, may be preferred in various contexts. In some contexts, it may be desirable to produce and/or preserve a dsRNA stem loop that is perfectly complementary (i.e., the complementary strands are perfect palindromes of each other and the dsRNA stem loop does not feature any mismatches, bulges, or interior loops).

Those of skill in the art will appreciate, however, that preferential alteration of the 5′ sequence of the dsRNA stem region (“miR” in “miR:miR*”) may disrupt complementarity between the 5′ and 3′ (“miR” and “miR*”) regions of the dsRNA stem. Thus, in such situations it will be desirable to ensure that complementarity is maintained and/or perfected between the 5′ and 3′ regions of the dsRNA stem. Complementarity may be maintained and/or perfected using a variety of approaches. In one example, a sequence may be employed that encodes only a 5′ (“miR”) region, a hairpin loop, and a priming site for priming the 3′ (“miR*”) region. In such circumstance, DNA polymerization may be used to extend the dsRNA stem, using the 5′ (“miR”) sequence as a template for forming a perfectly complementary 3′ (“miR*”) region of the pri-miRNA. In this regard, a variety of alternatively cloning and/or PCR schemes, easily determined by those skilled in the art, may be employed.

TABLE 1
Nucleotide Sequences of Exemplary Palindrome Optimized pri-miRNA Scaffolds According to Certain
Embodiments of the Present Disclosure

| Sequence | Sequence | Nucleotide Sequence of Palindrome Optimized Pri-miRNA Scaffolds Based |
|---|---|---|
| Identifier | Description | upon Naturally Occurring Pri-miRNA Sequences |
| SEQ ID NO: 1 | Let-7a | GATTCCTTTTCACCATTCACCCTGGATGTTCTCTTCACTGTGGGATGAGGTAGTAGGTTGTACAGTTTTAGGGT |
| | | CACACCCACCACTGGGAGATAACTGTACAACCTACTGTCTCATCCTAACGTGATAGAAAAGTCTGCATCCAGGC |
| | | GGTCTGATAGAAAGTC |
| SEQ ID NO: 2 | miR-16 | TTCTTTTTATTCATAGCTCTTATGATAGCAATGTCAGCAGTGCCTTAGCAGCACAGTGACTATTGGCGTTAAGA |
| | | TTCTAAAATTATCTCCAGTAGTCACTGTGCTGCTGAAGTAAGGTTGACCATACTCTACAGTTGTGTTTTAATGT |
| | | ATATTA |
| SEQ ID NO: 3 | miR-21 | ACCATCGTGACATCTCCATGGCTGTACCACCTTGTCGGGTAGCTTATCGACTGGTGTTGACTGTTGAATCTCAT |
| | | GGCAACACCAGTCGATGGGCTGTCTGACATTTTGGTATCTTTCATCTGACCATCCATATCC |
| SEQ ID NO: 4 | miR-22 | CATTTTCCCTCCCTTTCCCTTAGGAGCCTGTTCCTCTCACGCCCTCACCTGGCTGAGCCGCAGTAGTTCTTCAG |
| | | CTGGCAGCTTTATGTCCTGACCCAGCTAAAGCTGCCAGTTGAAGAACTACTGCCCTCTGCCCCTGGCTTCGAGG |
| | | AGGAGGAGGAGCTGCTTTCCCCATCAT |
| SEQ ID NO: 5 | miR-134 | TTCCGGAAGAGATGTTGGTGCCAGCACCATTCAGGGTGTGTGACTAGTTGTCCCACAGGGTCGTGCACTCTGTT |
| | | CACCCTGTGGGACAACTAGTCACACACCCTCAGCATCAATTCCACTCAAAGAAGACTTTCCA |
| SEQ ID NO: 6 | miR-370 | CTCATTCTACAAACCGTACAAGTCGGGGCACAAGACAGAGAAGCCAGGTTCCGTCTCAGCAGTCACCACAGCTC |
| | | ACGAGTGACTGCTGGGATGGAACCTGGTCTGTCTGTCTGTCTAACACCAGAGCTCGGGCGCTGCTG |
| SEQ ID NO: 7 | miR-371a | GCGATCGCCGCCTTGCCGCATCCCCTCAGCCTGTGGCACTCAAACTGTGGCGGCACTTTCTGCTCTCTGGTGAA |
| | | AGTGCCGCCATAGTTTGAGTGTTACAGCTTGAGAAGACTCAACCTGCGGAGAAGATA |
| SEQ ID NO: 8 | miR-371b | CAATCAAAATGGTATCTTCTCCGCAGGTTGAGTCTTCTCAAGCGGTTGCACTCAAACGATGGAGGCACTTTCAC |
| | | CAGAGAGCAGAAAGTGCCTCCATCGTTTGAGTGACACAGGCTGAGGGGATGCGGCAAGGCGGCGATCGCAAGTG |
| | | GAAG |
| SEQ ID NO: 9 | miR-372 | AAATTTCTTGGCCGGGGCTCTTGCAGATGGAGCTGCTCACCCTGTGGCTCTCAAATGTCGAGCACTGTTCTGAT |
| | | GTCCAAGTGGACAGTGCTCGACATTTGAGAGTCACCGGTGACGCCCATATCAACGGATGCCGTGGAGCTCGGTC |
| | | TTCT |
| SEQ ID NO: 10 | miR-373 | AGAAAGTCACAGTGATGGCAGATCCTCGCGAGGAGCTCATACTGGGACAACTCAAAATCGAAGCGCTGTCCTTT |
| | | TTGTCTGTACTGGACAGTGCTTCGATTTTGGGTTGTCCCTGTTTGAGTAGGGCATCACGAACCATCCTGCTTCA |
| | | AGGGAGCC |
| SEQ ID NO: 11 | miR-888 | GCGACAGCACCTCCACAATTAGCCATGTTGTGGGCAGTGCTCTACTCAAAGAGCTGTCAGTCACTTAGATTACA |
| | | TGTGACTGACAGCTCTTTGGGTGAAGGAAGGCTCACCAAGTACACTTTTGTGGTGGTCCTCAGACTG |
| SEQ ID NO: 12 | miR-290a | CCTCCTGGTAAATGTTTTAACGTCTGGACCGCCTCATCTTGCGGTACTCAAACTAGGCGGCACTTTATTTTTTT |
| | | CTTTAAAAAGTGCCGCCTAGTTTGAGTACCGCCGGTTGAGAAAACGGCTATCTGGCACATTTACCAGCCCTG |
| SEQ ID NO: 13 | miR-290b | GGGCTGGTAAATGTGCCAGATAGCCGTTTTCTCAACTGGCGGGACTTCAAACTAGTCGGCACTTTTTAAAGAAA |
| | | AAAAAAGTGCCGACTAGTTTGAAGTCCCGCCAGATGAGGCGGTCCAGACGTTAAAACATTTACCAGGAGA |
| SEQ ID NO: 14 | 291a | TGTGCGTCCTGTCGCTGCTTGGAGCTGTTACACCTATGTAGCGGGCATCAAAGTGGAGGCCCTCTCTTGAGCCT |
| | | GAATGAGAAAGTGCTTCCACTTTGTGTGCCACTGCATGAGGAAAACATCATAGCTTTGCATCTGACGCATCG |
| SEQ ID NO: 15 | miR-291b | GAGGAGCTGAGCCTCAGGTCTTGGAAGTTGGAACATACAGTGTCAGTCAAAGTGGAGGCCCTCTCCGCGGCTTG |
| | | GCGGGAGAGGGCCTCCATTTTGACTGTCTCTGTGTGTACATGGCGTCTGAAAAGCCGGCTGCAGCTGCA |
| SEQ ID NO: 16 | miR-292a | GTGCATTGTAGATTGTTCTTAAGCTTGACCTCCAGCCTGTGATACTCAAAACCTGGCGGCACTGTTCGATTTTC |
| | | ATCGGAAGAACAGTGCCGCCAGGTTTTGAGTGTCACAGGTTGAGAACTCAAAACGGCTAAGAACTAAGAATTAA |
| SEQ ID NO: 17 | miR-292b | TTAATTCTTAGTTCTTAGCCGTTTTGAGTTCTCAACCGGTGACACTCAAACTGGTGGCACCTTTCTTCCGATGA |
| | | AAATCCAAAGGTGCCACCAGTTTGAGTATCACAGGCTGGAGGTCAAGCTTAAGAACAATCTACAATGCACCT |
| SEQ ID NO: 18 | miR-293 | TTTATTACCACAGCAGGTCCCTGTGGAAATGCTTCAATCTGTGGTACTCAAACTCTGTGACATCTTGCTTTGTA |
| | | AGAAGATGTCGCAGAGTTTGAGTGTTGCCGATTGAGAAACTGCAGTTGGCCTAAGTGTTGCATCATTT |
| SEQ ID NO: 19 | miR-294 | TTCAGTTGTAGGCATGTTTGGATCTAGCGAAATTCCATATAGCCATACTCAAAATGGAGGCCCTGTCTAAGCTT |
| | | TTAAGTGGACAGGGCTTCCATTTTGAGTGTTGCCATGTGGAGAAAGCATCGGAACTTCAATCAACCAGAGACTC |
| SEQ ID NO: 20 | miR-295 | GGAACTTCAATCAACCAGAGACTCCTTGCTTGCTCATCTTGGTGAGACTCAAACGTGGTGGCACTCTTCGGACT |
| | | GTACATAGAGAGTGCTACTACGTTTGAGTCTCTCCTGTGGGCACCATTCACAGGTCGGCTGCTTTCATGCATGT |
| | | G |
| SEQ ID NO: 21 | miR-296 | ACACAGAACAGCCTATGTGGAAGGTGACAAGAGGGCCTTTCTGGAGGGCCTCCACTCAATCCTGTTGTGCTCGC |
| | | TTCAAAGGATTGGGTGGAGGCTCTCCTGAAGGTGTCCGCAGAGCGTCGCATGGTAAGTAGTCCCTTGGG |
| SEQ ID NO: 22 | artificial | CTAGCACGTAAAGCCGCGCAGAGTACGTCAGCATCGCCTACGTACTGCCATTACGTTAACTGCCA |
| | | ATCGTAGCTAGCTGGCAGTTAACGTAATGGCAGTACGTAGGCGATGTATTTATATCGGCCTGCATTATAGTATG |
| | | TA |
| SEQ ID NO: 23 | artificial | CTAGCACGTAAAGCCGCGCAGAGTACGTCAGC ATCGCCTACGT ACTGCCATTACGTTAACTGCCA |
| | | TAGTCGACGTAG TGGCAGTTAACGTAATGGCAGTACGTAGGCGAT |
| | | GTATTTATATCGGCCTGCATTATAGTATGTA |
| SEQ ID NO: 24 | artificial | CTAGCACGTAAAGCCGCGCAGAGTACGTCAGC ATCGCCTACGT ACTGCCATTACGTTAACTGCCA |
| | | CTAGAGCTGAAT TGGCAGTTAACGTAATGGCAGTACGTAGGCGAT |
| | | GTATTTATATCGGCCTGCATTATAGTATGTA |
| SEQ ID NO: 25 | artificial | TCGTACGACTAGCGTTCATCAGGTGCATCTAC CACCGCGTACT TCTATGATCTCTATCATTAGTC |
| | | ATCGTAGCTAGC GACTAATGATAGAGATCATAGAAGTACGCGGTG |
| | | GCTCACGTACGTACGTACCTCGTCAAGACTG |
| SEQ ID NO: 26 | artificial | TCGTACGACTAGCGTTCATCAGGTGCATCTAC CACCGCGTACT TCTATGATCTCTATCATTAGTC |
| | | TAGTCGACGTAG GACTAATGATAGAGATCATAGAAGTACGCGGTG |
| | | GCTCACGTACGTACGTACCTCGTCAAGACTG |
| SEQ ID NO: 27 | artificial | TCGTACGACTAGCGTTCATCAGGTGCATCTAC CACCGCGTACT TCTATGATCTCTATCATTAGTC |
| | | CTAGAGCTGAAT GACTAATGATAGAGATCATAGAAGTACGCGGTG |
| | | GCTCACGTACGTACGTACCTCGTCAAGACTG |

### Non-Native Pri-microRNA (Pri-miRNA) Libraries and Methods for Making Non-Native Pri-miRNA Libraries

Within certain embodiments, the present disclosure provides pri-microRNA (pri-miRNA) libraries and methods for the design and preparation of such pri-miRNA libraries, which pri-miRNA libraries contain one or more non-native pri-microRNAs that comprise a pri-microRNA scaffold and one or more polynucleotide fragments, each of which polynucleotide fragments includes a core nucleotide sequence that is flanked on its 5′ and 3′ ends with nucleotide sequence conferring additional binding specificity to the polynucleotide fragments. Regardless of the precise sequence of a pri-miRNA scaffold, it will be understood that the target nucleic acid specificity of any given miRNA is determined, principally, by (1) a 6 nucleotide “seed sequence” and (2) a nucleotide sequence that is 2-7 nucleotides from the 5′ end of a mature miRNA.

As described in further detail herein, the presently disclosed pri-miRNA libraries include DNA sequences encoding distinct non-native pri-miRNA based upon a selected pri-miRNA scaffold having a defined nucleotide sequence in combination with miRNA seed and flanking sequences (i.e., having a total length of from 6 to 23 nucleotides). Regardless of the precise sequence of the miRNA scaffold, however, it will be understood that each member pri-miRNA of a pri-miRNA library will incorporate a miRNA scaffold that obeys the structural requirements of miRNA-biogenesis pathway, as discussed in detail herein and as otherwise known in the art.

To ensures that pri-miRNAs having all possible combinations of seed sequences and flanking sequences are represented in the libraries, the nucleotide sequences of the pri-miRNA seed or flanking sequences are randomized in order to achieve pri-miRNA libraries having a complexity of distinct pri-miRNA sequences of from 10⁴ non-native pri-miRNA to 10⁹ non-native pri-miRNA.

In certain applications, it will be desirable to randomize only the seed sequences and maintain a single, defined flanking sequence. In such a case, libraries are prepared having a complexity of from 10⁴ non-native pri-miRNA to 10⁵ non-native pri-miRNA. That is, assuming a seed sequence length of 6 nucleotides, the library will have a complexity of 4⁶=4,096 in which all possible seed sequences are included.

In other applications, it will be desirable to randomize both the seed sequences and one or more nucleotides of the flanking sequences. In such cases, libraries are prepared having complexities of from 10⁵ non-native pri-miRNA to 10⁹ non-native pri-miRNA. That is, assuming a seed sequence length of 6 nucleotides, individual libraries will have complexities of 10⁵ to 10⁹ (i.e. for a fully-randomized 6 nucleotide seed sequence and 1 fully-flanking nucleotide, a complexity of 4⁷=16,384 is required and for a fully-randomized 6 nucleotide seed sequence and 9 fully-randomized flanking nucleotides, a complexity of 4¹⁵=1,073,741,824 is required). It will be understood that, for certain applications, libraries having partially-randomized seed or flanking sequences may be desired. The present disclosure, therefore, contemplates libraries having complexities of at least 10⁵, at least 10⁶, at least 10⁷, at least 10⁸, or at least 10⁹ distinct non-native pri-miRNAs.

Methodology for the production of complex nucleic acid libraries that may be adapted for use in the preparation of pri-miRNA libraries according to the present disclosure have been previously described. Pri-miRNA libraries can, for example, be prepared using highly parallel in situ oligonucleotide synthesis methodologies as described in Cleary et al., Nature Methods 1(3):241 (2004), which uses in situ microarray DNA synthesis to generate complex oligonucleotide populations that can be used directly or cloned into a suitable vector.

### Methods for Screening Non-Native Pri-microRNA (Pri-miRNA) Libraries

Within certain embodiments, the present disclosure provides methods for screening the non-native pri-miRNA libraries described herein.

Conventional methodologies for identifying target mRNAs for a given miRNA include biochemical assays, such as pull-down assays, in which the corresponding miRNA-processing proteins are enriched by their affinity for the miRNA. Target mRNAs are subsequently identified by detecting/sequencing mRNAs bound to those processing proteins. For example, by pulling down members of the Argonaute (Ago) protein family, several mRNA targets have been identified, including targets for a specific miRNA, miR-124.

HITS-CLIP (high-throughput sequencing to crosslinking immunoprecipitation) technologies have been employed to develop a genome-wide map of interactions between the neuron-specific splicing factor Nova and RNA in the mouse brain. Thus, HITS-CLIP provides a means for identifying functional protein-RNA interactions in vivo. HITS-CLIP has also been used to decode a map of miRNA-binding sites to brain mRNA transcripts by covalently crosslinking native Ago protein-RNA complexes in mouse brain. By simultaneously defining Ago-miRNA and Ago-mRNA interactions and bioinformatically assessing whether these mRNAs contain a miRNA-binding site, genome-wide interaction maps for miR-124 have been validated and additional maps for miRNAs present in P13 mouse brain have been generated.

A shortcoming of these and other methodologies known in the art, which is overcome by the methods of the present disclosure, is the strict reliance on the identification of specific structural interactions without consideration of the effect, if any, of those interactions on the resulting phenotype of a cell expressing one or more pri-miRNAs. Moreover, such approaches do not permit the identification of miRNAs that work in concert to affect one or more cellular phenotype.

The pri-miRNA libraries and the use of those libraries to screen for individual pri-miRNAs, or groups of miRNAs, that effect a desired cellular function or result in a desired cellular phenotype. Thus, the methods disclosed herein rely on phenotypic outcome without requiring a pre-conception of target specificity.

Once identified, the specificity of one or more non-native pri-miRNAs can be enhanced by applying methodologies employing sequential expression, selection, amplification, and mutation, such as, for example, in a manner analogous to the in vitro selection or in vitro evolution methodologies described in the art, including, for example, via systematic evolution of ligands by exponential (SELEX) enrichments as has been described for the engineering of nucleic acid aptamers having improved binding affinity or specificity for a given molecular target. SELEX is a combinatorial molecular biology technique for that permits the production of single-stranded DNA or RNA oligonucleotides that specifically bind to a target ligand or ligands. Oliphant et al., Mol. Cell Biol. 9:2944-2949 (1989); Tuerk & Gold, Science 249:505-510 (1990); and Ellington & Szostak, Nature 346:818-822 (1990). See, also, Blackwell & Weintraub, Science 250:1104-1110 (1990) (describing a related methodology “selected and amplified binding site” (SAAB)) and Wright et al., Mol. Cell Biol. 11:4104-4110 (1991) (describing a related methodology “cyclic amplification and selection of targets” (CASTing),

Briefly, SELEX the process begins with the synthesis of a very large oligonucleotide library consisting of randomly generated sequences of fixed length flanked by constant 5′ and 3′ ends that serve as primers. For a randomly generated region of length n, the number of possible sequences in the library is 4ⁿ (n positions with four possibilities (A,T,C,G) at each position). The sequences in the library are exposed to the target ligand, which may be a protein or a small organic compound—and those that do not bind the target are removed, usually by affinity chromatography. The bound sequences are eluted and amplified by PCR to prepare for subsequent rounds of selection in which the stringency of the elution conditions is increased to identify the tightest-binding sequences. An advancement on the original method allows an RNA library to omit the constant primer regions, which can be difficult to re-move after the selection process because they stabilize

However, unlike aptamers, which are entirely random and possess tertiary structures that bind target proteins, miRNA derive their function from passing through each canonical step of the micro-RNA biogenesis pathway and expressing seed sequences in specific patterns. Using a library of randomized miRNA as a starting point, multiple rounds of selection, expression, and mutation could be used to generate novel miRNAs that induce desired cell states. Over multiple rounds of selection, sets of de novo miRNA may emerge that facilitate very fast and specific differentiation by triggering temporally ordered changes in gene expression that differ as a function of cellular signaling, and/or constitutively express appropriate miRNA-sequences. Once these sequences were determined, they could be expressed genomically for certain purposes, or transiently so as not to alter the genome. This would be highly desirable for research in neurobiology, where these miRNA may be used to generate populations of defined neural cells. These neural cells could be used to build model systems of neural circuitry in vitro, which have many applications, from disease research, to developmental biology, and the investigation the molecular correlates of models of human behaviors such as memory, learning, or perception.

### Vectors and Cells for Expressing Non-Native Pri-miRNAs

Within certain embodiments, this disclosure provides vectors comprising one or more pri-miRNAs, which vectors are configured for the expression of the one or more pri-miRNAs. Within related embodiments, the present disclosure provides cells comprising one or more vector that is configured for the expression of one or more pri-miRNAs.

Thus, the presently disclosed vectors include nucleic acid delivery vectors that may be non-specific with respect to the cell type to which the pri-miRNAs are delivered. The vectors described herein may, but need not be, configured for target cell-specific delivery of one or more pri-miRNAs to achieve target cell specificity and, consequently, the desired regulation, promotion, normalization, restoration, inhibition, or modulation of a desired cellular phenotype (e.g., cell maintenance, survival, growth/proliferation, differentiation, or death) within the targeted cell.

Within certain aspects of these embodiments, the present disclosure provides vectors for the expression of a pri-miRNA within a target cell. The expression vectors disclosed herein comprise: (1) a transcriptional promoter that is activated in response to one or more factors each of which is produced within a target cell and (2) nucleotide sequences encoding one or more pri-miRNAs that are operably linked to and under regulatory control of the transcriptional promoter, wherein the one or more pri-miRNAs can regulate, promote, normalize, restore, inhibit, or modulate a desired cellular phenotype (e.g., cell maintenance, survival, growth/proliferation, differentiation, or death) within the targeted cell.

Within related aspects of these embodiments, the transcriptional promoter can be activated in a target cell that is associated with a disease or condition or a target cell, such as a stem cell (e.g., an embryonic stem cell (ESC) or an induced pluripotent stem cell (iPSC)) that is to be differentiated along a desired cell lineage. Transcriptional activation can be achieved by the action of one or more factors that are produced in the target cell, such as a cancer cell, a precancerous cell, a dysplastic cell, or a cell that is infected with an infectious agent. Target cells may also include a hematopoietic cell, an adipose cell, an eye cell, a brain cell, a liver cell, a colon cell, a lung cell, a pancreas cell, a breast cell, a prostate cell, a colorectal cell, or a heart cell.

Suitable transcriptional promoters that may be employed in vectors for the expression of one or more pri-miRNAs include, for example, the p21^{cip1/waf1} promoter, the p27^{kip1} promoter, the p57^{kip2} promoter, the TdT promoter, the Rag-1 promoter, the B29 promoter, the Blk promoter, the CD19 promoter, the BLNK promoter, and the λ5 promoter, which transcriptional promoter is responsive to activation by one or more transcription factors such as an EBF3, O/E-1, Pax-5, E2A, p53, VP16, MLL, HSF1, NF-IL6, NFAT1, AP-1, AP-2, HOX, E2F3, and/or NF-κB transcription factor, and which transcriptional activation induces the expression of a nucleic acid that encodes a pri-miRNA as described in further detail herein.

Target cells include human cells that are infected with an infectious agent, such as a virus, including, for example, a herpes virus, a polio virus, a hepatitis virus, a retrovirus virus, an influenza virus, or a rhino virus. In the case of target cells that are infected with an infectious agent, the vector expressing the pri-miRNAs may employ a transcriptional promoter that is activated by a factor expressed by the infectious agent to, thereby, induce the expression of a nucleic acid encoding the pri-miRNA.

Within other aspects of these embodiments, the vector may be configured to non-specifically deliver a nucleic acid encoding a pri-miRNA to a target cell as well as a non-target cell, wherein the vector comprises a transcriptional promoter that is responsive to a transcription factor that is specifically or preferentially expressed in the target cell (e.g., a cell that is associated with a disease or other condition or a cell that is in a particular stage of differentiation), but is not expressed in the non-target cell and wherein the vector comprises a nucleic acid that encodes a pri-miRNA that can regulate, promote, normalize, restore, inhibit, or modulate a desired cellular phenotype (e.g., cell maintenance, survival, growth/proliferation, differentiation, or death) within the targeted cell.

Thus, certain aspects, the various embodiments provided by the present disclosure include:

(1) vectors that are configured for the expression of one or more pri-miRNAs within a target cell, such as a cell that is associated with a disease, a condition, or a stage of differentiation, wherein the vectors comprise:
(a) a transcriptional promoter that is activated in response to one or more factors that are produced within a target cell and
(b) a nucleic acid that is operably linked to and under regulatory control of the transcriptional promoter, wherein the nucleic acid encodes one or more pri-miRNA that, when processed in vivo to a mature miRNA, can regulate, promote, normalize, restore, inhibit, or modulate a desired cellular phenotype (e.g., cell maintenance, survival, growth/proliferation, differentiation, or death) within the targeted cell.

Thus, in other aspects, the various embodiments provided by the present disclosure include: target cells comprising one or more vectors as described herein that are configured for the expression of one or more pri-miRNAs within the target cell, such as a cell that is associated with a disease, a condition, or a stage of differentiation, wherein the target cells are susceptible to regulation, promotion, normalization, restoration, inhibition, or modulation of a desired cellular phenotype (e.g., cell maintenance, survival, growth/proliferation, differentiation, or death) in response to mature miRNAs that result from the in vivo processing of the one or more pri-miRNAs that are delivered to the target cell and expressed by the one or vectors disclosed herein.

The presently disclosed pri-miRNA expression vectors may be used in methods for the treatment of cancer, infectious disease, or other conditions as well as in methods for the differentiation of stem cells, such as ESC or iPSC, into a cell of a desired lineage.

Thus, the present disclosure provides vectors effectuating one or more cellular activity thereby the growth, survival, or differentiation of a broad range of cells, including those that are associated with a disease or other condition that similarly comprises (1) a non-specific nucleic acid delivery vector and (2) an expression construct comprising (a) a target cell specific transcriptional promoter and (b) a nucleic acid that encodes a therapeutic pri-miRNA. Each of these aspects of the presently disclosed systems are described in further detail herein.

Within certain embodiments, provided herein are systems for effectuating the growth, survival, or differentiation of target cells, which systems comprise: (1) a non-specific nucleic acid delivery vector and (2) an expression construct comprising: (a) a transcriptional promoter, which transcriptional promoter is activated in target cells but not in normal, non-target cells, and (b) a nucleic acid that is under the control of the transcriptional promoter, which nucleic acid encodes a pri-miRNA that can reduce, prevent, or eliminate the growth or survival of a target cell or can promote the differentiation of a target cell.

In certain aspects of these embodiments wherein the human target cell is a cancer cell, such as a brain cancer cell, a prostate cancer cell, a lung cancer cell, a colorectal cancer cell, a breast cancer cell, a liver cancer cell, a hematologic cancer cell, and a bone cancer cell, the transcriptional promoter can include at least a transcription factor binding site (i.e., a response element) of the p21^{cip1/waf1} promoter, the p27^{kip1} promoter, the p57^{kip2} promoter, the TdT promoter, the Rag-1 promoter, the B29 promoter, the Blk promoter, the CD19 promoter, the BLNK promoter, and/or the λ5 promoter, which transcriptional promoter is responsive to activation by one or more transcription factors such as an EBF3, O/E-1, Pax-5, E2A, p53, VP16, MLL, HSF1, NF-IL6, NFAT1, AP-1, AP-2, HOX, E2F3, and/or NF-κB transcription factor, and which transcriptional activation induces the expression of a nucleic acid that encodes one or more pri-miRNAs that can regulate, promote, normalize, restore, inhibit, or modulate a desired cellular phenotype (e.g., cell maintenance, survival, growth/proliferation, differentiation, or death) within the targeted cell.

As used herein, the term “transcriptional promoter” refers to a promoter is a region of DNA that initiates transcription of a particular gene. Promoters are located near the transcription start sites of genes, on the same strand and upstream on the DNA (towards the 3′ region of the anti-sense strand, also called template strand and non-coding strand). Promoters can be about 100-1000 base pairs long. For the transcription to take place, the enzyme that synthesizes RNA, known as RNA polymerase, must attach to the DNA near a gene. Promoters contain specific DNA sequences and response elements that provide a secure initial binding site for RNA polymerase and for proteins called transcription factors that recruit RNA polymerase. These transcription factors have specific activator or repressor sequences of corresponding nucleotides that attach to specific promoters and regulate gene expressions. The process is more complicated, and at least seven different factors are necessary for the binding of an RNA polymerase II to the promoter. Promoters represent critical elements that can work in concert with other regulatory regions (enhancers, silencers, boundary elements/insulators) to direct the level of transcription of a given gene.

Eucaryotic transcriptional promoters comprise a number of essential elements, which collectively constitute a core promoter (i.e., the minimal portion of a promoter that is required to initiate transcription). Those elements include (1) a transcription start site (TSS), (2) an RNA polymerase binding site (in particular an RNA polymerase II binding site in a promoter for a gene encoding a messenger RNA), (3) a general transcription factor binding site (e.g., a TATA box having a consensus sequence TATAAA, which is a binding site for a TATA-binding protein (TBP)), (4) a B recognition element (BRE), (5) a proximal promoter of approximately 250 bp that contains regulatory elements, (6) transcription factor binding sites (e.g., an E-box having the sequence CACGTF, which is a binding site for basic helix-loop-helix (bHLH) transcription factors including BMAL11-Clock nad cMyc), and (7) a distal promoter containing additional regulatory elements. As used herein, the term “transcriptional promoter” is distinct from the term “enhancer,” which refers to a regulatory element that is distant from the transcriptional start site.

Eucaryotic promoters are often categorized according to the following classes: (1) AT-based class, (2) CG-based class, (3) ATCG-compact class, (4) ATCG-balanced class, (5) ATCG-middle class, (6) ATCG-less class, (7) AT-less class, (8) CG-spike class, (9) CG-less class, and (10) ATspike class. See, Gagniuc and Ionescu-Tirgoviste, BMC Genomics 13:512 (2012).

Eucaryotic promoters can be “unidirectional” or “bidirectional.” Unidirectional promoters regulate the transcription of a single gene and are characterized by the presence of a TATA box. Bidirectional promoters are short (<1 kbp), intergenic regions of DNA between the 5′ ends of genes in a bidirectional gene pair (i.e., two adjacent genes coded on opposite strands having 5′ ends oriented toward one another. Bidirectional genes are often functionally related and because they share a single promoter, can be co-regulated and co-expressed. Unlike unidirectional promoters, bidirectional promoters do not contain a TATA box but do contain GpC islands and exhibit symmetry around a midpoint of dominant Cs and As on one side and Gs and Ts on the other. CCAAT boxes are common in bidirectional promoters as are NRF-1, GABPA, YY1, and ACTACAnnTCCC motifs.

Transcriptional promoters often contain two or more transcription factor binding sites. Thus, the efficient expression of a nucleic acid that is downstream of a promoter having multiple transcription factor binding sites typically requires the cooperative action of multiple transcription factors. Accordingly, the specificity of transcriptional regulation, and hence expression of an associated nucleic acid, can be increased by employing transcriptional promoters having two or more transcription factor binding sites.

As used herein, the term “transcription factor” refers to sequence-specific DNA-binding factors that bind to specific sequences within a transcriptional promoter thereby regulating the transcription of a nucleic acid that is in operable proximity to and downstream of the promoter. Transcription factors include activators, which promote transcription, and repressors, which block transcription by preventing the recruitment or binding of an RNA polymerase. Transcription factors typically contain (1) one or more DNA-binding domains (DBDs), which facilitate sequence specific binding to a cognate transcription factor binding site (a/k/a response element) within a transcriptional promoter; (2) one or more signal-sensing domains (SSDs), which includes ligand binding domains that are responsive to external signals; and (3) one or more transactivation domains (TADs), which contain binding sites for other proteins, including transcription coregulators.

As used herein, the term “transcription factor” refers exclusively to those factors having one or more DBDs and is not intended to include other regulatory proteins such as coactivators, chromatin remodelers, histone acetylases, deacetylases, kinases, and methylases, which no not contain DBDs.

A wide variety of both non-viral and viral nucleic acid delivery vectors are well known and readily available in the art and may be adapted for use for the non-specific cellular delivery of the expression constructs disclosed herein. See, for example, Elsabahy et al., Current Drug Delivery 8(31:235-244 (2011) for a general description of viral and non-viral nucleic acid delivery methodologies. The successful delivery of a nucleic acid into mammalian cells relies on the use of efficient delivery vectors. Viral vectors exhibit desireable levels of delivery efficiency, but often also exhibit undesireable immunogenicity, inflammatory reactions, and problems associated with scale-up, all of which can limit their clinical use. The ideal vectors for the delivery of a nucleic acid are safe, yet ensure nucleic acid stability and the efficient transfer of the nucleic acid to the appropriate cellular compartments.

Non-limiting examples of non-viral and viral nuclic acid delivery vectors are described herein and disclosed in scientific and patent literature. More specifically, the presently disclosed systems may employ one or more liposomal vectors, viral vectors, nanoparticles, polyplexesm dendrimers, each of which has been developed for the non-specific delivery of nucleic acids, can be adapted for the non-specific delivery of the expression constructs described herein, and can be modified to incorporate one or more agents for promoting the targeted delivery of a system to a target cell of interest thereby enhancing the target cell specificity of the presently disclosed systems.

An expression cassette may be incorporated within and/or associated with a lipid membrane, a lipid bi-layer, and/or a lipid complex such as, for example, a liposome, a vesicle, a micelle and/or a microsphere. Suitable methodology for preparing lipid-based delivery systems that may be employed with the expression constructs of the present disclosure are described in Metselaar et al., Mini Rev. Med. Chem. 2(4):319-29 (2002); O'Hagen et al., Expert Rev. Vaccines 2(2):269-83 (2003); O'Hagan, Curr. Durg Targets Infect. Disord. 1(3):273-86 (2001); Zho et al., Biosci Rep. 22(2):355-69 (2002); Chikh et al., Biosci Rep. 22(2):339-53 (2002); Bungener et al., Biosci. Rep. 22(2):323-38 (2002); Park, Biosci Rep. 22(2):267-81 (2002); Ulrich, Biosci. Rep. 22(2):129-50; Lofthouse, Adv. Drug Deliv. Rev. 54(6):863-70 (2002); Zhou et al., J. Immunother. 25(4):289-303 (2002); Singh et al., Pharm Res. 19(6):715-28 (2002); Wong et al., Curr. Med. Chem. 8(9):1123-36 (2001); and Zhou et al., Immunomethods 4(3):229-35 (1994). Midoux et al., British J. Pharmacol 157:166-178 (2009) describe chemical vectors for the delivery of nucleic acids including polymers, peptides and lipids. Sioud and Sorensen, Biochem Biophys Res Commun 312(4):1220-5 (2003) describe cationic liposomes for the delivery of nucleic acids.

Due to their positive charge, cationic lipids have been employed for condensing negatively charged DNA molecules and to facilitate the encapsulation of DNA into liposomes. Cationic lipids also provide a high degree of stability to liposomes. Cationic liposomes interact with a cell membrane and are taken up by a cell through the process of endocytosis. Endosomes formed as the results of endocytosis, are broken down in the cytoplasm thereby releasing the cargo nucleic acid. Because of the inherent stability of cationic liposomes, however, transfection efficiencies can be low as a result of lysosomal degradation of the cargo nucleic acid.

Helper lipids (such as the electroneutral lipid DOPE and L-a-dioleoyl phosphatidyl choline (DOPC)) can be employed in combination with cationic lipids to form liposomes having decreased stability and, therefore, that exhibit improved transfection efficiencies. These electroneutral lipids are referred to as fusogenic lipids. See, Gruner et al., Biochemistry 27(8):2853-66 (1988) and Farhood et al., Biochim Biophys Acta 1235(2):289-95 (1995). DOPE forms an HII phase structure that induces supramolecular arrangements leading to the fusion of a lipid bilayer at a temperature greater than 5° C. to 10° C. The incorporation of DOPE into liposomes also helps the formation of HII phases that destabilize endosomal membranes.

Cholesterol can be employed in combination with DOPE liposomes for applications in which a liposomal vector is administered intravenously. Sakurai et al., Eur J Pharm Biopharm 52(2):165-72 (2001). The presence of one unsaturation in the acyl chain of DOPE is a crucial factor for membrane fusion activity. Talbot et al., Biochemistry 36(19):5827-36 (1997).

Fluorinated helper lipids having saturated chains, such as DF4C11PE (rac-2,3-Di[11-(F-butyl)undecanoyl) glycero-1-phosphoethanolamine) also enhance the transfection efficiency of lipopolyamine liposomes. Boussif et al., J Gene Med 3(2):109-14 (2001); Gaucheron et al., Bioconj Chem 12(6):949-63 (2001); and Gaucheron et al., J Gene Med 3(4):338-44 (2001).

The helper lipid 1,2-dioleoyl-3-trimethylammonium-propane (DOTAP) enhances efficient of in vitro cell transfection as compared to DOPE lipoplexes. Prata et al., Chem Commun 13:1566-8 (2008). Replacement of the double bond of the oleic chains of DOPE with a triple bond as in Distear-4-ynoyl L-a-phosphatidylethanolamine [DS(9-yne)PE] has also been shown to produce more stable lipoplexes. Fletcher et al., Org Biomol Chem 4(2):196-9 (2006).

Amphiphilic anionic peptides that are derived from the N-terminal segment of the HA-2 subunit of influenza virus haemagglutinin, such as the IFN7 (GLFEAIEGFIENGWEGMIDGWYG) and E5CA (GLFEAIAEFIEGGWEGLIEGCA) peptides, can be used to increase the transfection efficiency of liposomes by several orders of magnitude. Wagner et al., Proc Natl Acad Sci U.S.A. 89(17):7934-8 (1992); Midoux et al., Nucl Acids Res. 21(4):871-8 (1993); Kichler et al., Bioconjug Chem 8(2):213-21 (1997); Wagner, Adv Drug Deliv Rev 38(3):279-289 (1999); Zhang et al., J Gene Med 3(6):560-8 (2001). Some artificial peptides such as GALA have been also used as fusogenic peptides. See, for example, Li et al., Adv Drug Deliv Rev 56(7):967-85 (2004) and Sasaki et al., Anal Bioanal Chem 391(8):2717-27 (2008). The fusogenic peptide of the glycoprotein H from herpes simplex virus improves the endosomal release of DNA/Lipofectamine lipoplexes and transgene expression in human cell (Tu and Kim, J Gene Med 10(6):646-54 (2008).

PCT Patent Publication No. WO 2002/044206 describes a class of proteins derived from the family Reoviridae that promote membrane fusion. These proteins are exemplified by the p14 protein from reptilian reovirus and the p16 protein from aquareovirus. PCT Patent Publication No. WO 2012/040825 describes recombinant polypeptides for facilitating membrane fusion, which polypepides have at least 80% sequence identity with the ectodomain of p14 fusion-associated small transmembrane (FAST) protein and having a functional myristoylation motif, a transmembrane domain from a FAST protein and a sequence with at least 80% sequence identity with the endodomain of p15 FAST protein. The '825 PCT further describes the addition of a targeting ligand to the recombinant polypeptide for selective fusion. The recombinant polypeptides presented in the '825 PCT can be incorporated within the membrane of a liposome to facilitate the delivery of nucleic acids. Fusogenix liposomes for delivering therapeutic compounds, including nucleic acids, to the cytoplasm of a mammalian cell, which reduce liposome disruption and consequent systemic dispersion of the cargo nucleic acid and/or uptake into endosomes and resulting nucleic acid destruction are available commercially from Innovascreen Inc. (Halifax, Nova Scotia, CA).

A wide variety of inorganic nanoparticles, including gold, silica, iron oxide, titanium, hydrogels, and calcium phosphates have been described for the delivery of nucleic acids and can be adapted for the delivery of the expression constructs described herein. See, for example Wagner and Bhaduri, Tissue Engineering 18(1):1-14 (2012) (describing inorganic nanoparticles for delivery of nucleic acid sequences); Ding et al., Mol Ther e-pub (2014) (describing gold nanoparticles for nucleic acid delivery); Zhang et al., Langmuir 30(3):839-45 (2014) (describing titanium dioxide nanoparticles for delivery of DNA oligonucleotides); Xie et al., Curr Pharm Biotechnol 14(10):918-25 (2014) (describing biodegradable calcium phosphate nanoparticles fro gene delivery); Sizovs et al., J Am Chem Soc 136(1):234-40 (2014) (describing sub-30 monodisperse oligonucleotide nanoparticles).

Among the advantages of inorganic vectors are their storage stability, low immunogenicity, and resistance to microbial attack. Nanoparticles of less than 100 nm can efficiently trap nucleic acids and allows its escape from endosomes without degradation. Inorganic nanoparties exhibit improved in vitro transfection for attached cell lines due to their high density and preferential location on the base of the culture dish. Quantum dots have been described that permit the coupling of nucleic acid delivery with stable fluorescence markers.

Hydrogel nanoparticles of defined dimensions and compositions, can be prepared via a particle molding process referred to as PRINT (Particle Replication in Non-wetting Templates), and can be used as delivery vectors for the expression constructs disclosed herein. Nucleic acids can be encapsulated in particles through electrostatic association and physical entrapment. To prevent the disassociation of cargo nucleic acids from nanoparticles following systemic administration, a polymerizable conjugate with a degradable, disulfide linkage can be employed.

The PRINT technique permits the generation of engineered nanoparticles having precisely controlled properties including size, shape, modulus, chemical composition and surface functionality for enhancing the targeting of the expression cassette to a target cell. See, e.g., Wang et al., J Am Chem Soc 132:11306-11313 (2010); Enlow et al., Nano Lett 11:808-813 (2011); Gratton et al., Proc Natl Acad Sci USA 105:11613-11618 (2008); Kelly, J Am Chem Soc 130:5438-5439 (2008); Merkel et al. Proc Natl Acad Sci USA 108:586-591 (2011). PRINT is also amenable to continuous roll-to-roll fabrication techniques that permit the scale-up of particle fabrication under good manufacturing practice (GMP) conditions.

Nanoparticles can be encapsulated with a lipid coating to improve oral bioavailability, minimize enzymatic degradation and cross blood brain barrier. The nanoparticle surface can also be PEGylated to improve water solubility, circulation in vivo, and stealth properties.

A wide variety of viral vectors are well known by and readily available to those of skill in the art, including, for example, herpes simplex viral vectors lentiviral vectors, adenoviral vectors, and adeno-associated viral vectors, which viral vectors can be adapted for use in the systems disclosed herein for the delivery of nucleic acids, in particular nucleic acids comprising an expression cassette for the target cell specific expression of a therapeutic protein.

The tropisms of natural or engineered viruses towards specific receptors are the foundations for constructing viral vectors for delivery of nucleic acids. The attachment of these vectors to a target cell is contingent upon the recognition of specific receptors on a cell surface by a ligand on the viral vector. Viruses presenting very specific ligands on their surfaces anchor onto the specific receptors on a cell. Viruses can be engineered to display ligands for receptors presented on the surface of a target cell of interest. The interactions between cell receptors and viral ligands are modulated in vivo by toll like receptors.

The entry of a viral vector into a cell, whether via receptor mediated endocytosis or membrane fusion, requires a specific set of domains that permit the escape of the viral vector from endosomal and/or lysosomal pathways. Other domains facilitate entry into nuclei. Replication, assembly, and latency determine the dynamics of interactions between the vector and the cell and are important considerations in the choice of a viral vector, as well as in engineering therapeutic cargo carrying cells, in designing cancer suicide gene therapies.

Herpes simplex virus (HSV) belongs to a family of herpesviridae, which are enveloped DNA viruses. HSV binds to cell receptors through orthologs of their three main ligand glycoproteins: gB, gH, and gL, and sometimes employ accessory proteins. These ligands play decisive roles in the primary routes of virus entry into oral, ocular, and genital forms of the disease. HSV possesses high tropism towards cell receptors of the nervous system, which can be utilized for engineering recombinant viruses for the delivery of expression cassettes to target cells, including senescent cells, cancer cells, and cells infected with an infectious agent. Therapeutic bystander effects are enhanced by inclusion of connexin coding sequences into the constructs. Herpes Simplex Virus vectors for the delivery of nucleic acids to target cells have been reviewed in Anesti and Coffin, Expert Opin Biol Ther 10(1):89-103 (2010); Marconi et al., Adv Exp Med Biol 655:118-44 (2009); and Kasai and Saeki, Curr Gene Ther 6(3):303-14 (2006).

Lentivirus belongs to a family of retroviridae, which are enveloped, single stranded RNA retroviruses and include the Human immunodeficiency virus (HIV). HIV envelope protein binds CD4, which is present on the cells of the human immune system such as CD4+ T cells, macrophages, and dendritic cells. Upon entry into a cell, the viral RNA genome is reverse transcribed into double-stranded DNA, which is imported into the cell nucleus and integrated into the cellular DNA. HIV vectors have been used to deliver the therapeutic genes to leukemia cells. Recombinant lentiviruses have been described for mucin-mediated delivery of nucleic acids into pancreatic cancer cells, to epithelial ovarian carcinoma cells, and to glioma cells, without substantial non-specific delivery to normal cells. Lentiviral vectors for the delivery of nucleic acids to target cells have been reviewed in Primo et al., Exp Dermatol 21(3):162-70 (2012); Staunstrup and Mikkelsen, Curr Gene Ther 11(5):350-62 (2011); and Dreyer, Mol Biotechnol 47(2):169-87 (2011).

Adenovirus is a non-enveloped virus consisting of a double-stranded, linear DNA genome and a capsid. Naturally, adenovirus resides in adenoids and may be a cause of upper respiratory tract infections. Adenovirus utilizes a cell's coxsackievirus and adenovirus receptor (CAR) for the adenoviral fiber protein for entry into nasal, tracheal, and pulmonary epithelia. Recombinant adenovirus can be generated that are capable of nucleic acid deliver to target cells. Replication-competent adenovirus-mediated suicide gene therapy (ReCAP) is in the clinical trials for newly-diagnosed prostate cancer. Adenovirus vectors for the delivery of nucleic acids to target cells have been reviewed in Huang and Kamihira, Biotechnol Adv. 31(2):208-23 (2013); Alemany, Adv Cancer Res 115:93-114 (2012); Kaufmann and Nettelbeck, Trends Mol Med 18(7):365-76 (2012); and Mowa et al., Expert Opin Drug Deliv 7(12):1373-85 (2010).

Adeno-associated virus (AAV) is a small virus that infects humans and some other primate species. AAV is not currently known to cause disease and consequently the virus causes a very mild immune response. Vectors using AAV can infect both dividing and quiescent cells and persist in an extrachromosomal state without integrating into the genome of the host cell. These features make AAV a very attractive candidate for creating viral vectors for use in the systems of the present disclosure. Adeno-associated virus (AAV) vectors for the delivery of nucleic acids to target cells have been reviewed in Li et al., J. Control Release 172(2):589-600 (2013); Hajitou, Adv Genet 69:65-82 (2010); McCarty, Mol Ther 16(10):1648-56 (2008); and Grimm et al., Methods Enzymol 392:381-405 (2005).

Polyplexes are complexes of polymers with DNA. Polyplexes consist of cationic polymers and their fabrication is based on self-assembly by ionic interactions. One important difference between the methods of action of polyplexes and liposomes and lipoplexes is that polyplexes cannot directly release their nucleic acid cargo into the cytoplasm of a target cell. As a result co-transfection with endosome-lytic agents such as inactivated adenovirus is required to facilitate escape from the endocytic vesicle made during particle uptake. Better understanding of the mechanisms by which DNA can escape from endolysosomal pathway (i.e., the proton sponge effect) has triggered new polymer synthesis strategies such as the incorporation of protonable residues in polymer backbone and has revitalized research on polycation-based systems. See, e.g., Parhamifar et al., Methods e-pub (2014); Rychgak and Kilbanov, Adv Drug Deliv Rev e-pub (2014); Jafari et al., Curr Med Chem 19(2):197-208 (2012).

Due to their low toxicity, high loading capacity, and ease of fabrication, polycationic nanocarriers exhibit substantial advantages over viral vectors, which show high immunogenicity and potential carcinogenicity and lipid-based vectors which cause dose dependent toxicity. Polyethyleneimine, chitosan, poly(beta-amino esters), and polyphosphoramidate have been described for the delivery of nucleic acids. See, e.g., Buschmann et al., Adv Drug Deliv Rev 65(9):1234-70 (2013). The size, shape, and surface chemistry of these polymeric nano-carriers can be easily manipulated.

Dendrimers are highly branched macromolecules having a spherical shape. The surface of dendrimer particles may be functionalized such as, for example, with positive surface charges (cationic dendrimers), which may be employed for the delivery of nucleic acids. Dendrimer-nucleic acid complexes are taken into a cell via endocytosis. Dendrimers offer robust covalent construction and extreme control over molecule structure and size. Dendrimers are available commercially from Dendritic Nanotechnologies Inc. (Priostar; Mt Pleasant, Mich.), who produce dendrimers using kinetically driven chemistry, which can be adapted fro the delivery of nucleic acids and can transfect cells at a high efficiency with low toxicity.

It will be understood that, while targeted delivey of a vector is not required by the systems of the present disclosure and that the targeted reduction, prevention, and/or elimination in the growth and/or survival of a target cell may be achieved by exploiting the intracellular transcriptional machinery of a target cell that is unique to the target cell, it may be desireable, depending upon the precise application contemplated, the incorporate into an otherwise non-specific delivery vector one or more components that facilitate the targeted delivery to a subset of cells at least some of which include a target cell that is susceptible to the growth and/or survival inhibition by the expression constructs of the present disclosure.

Vectors can be administered to a human patient by themselves or in pharmaceutical compositions where they are mixed with suitable carriers or excipient(s) at doses to treat or ameliorate a disease or condition as described herein. Mixtures of these systems can also be administered to the patient as a simple mixture or in pharmaceutical compositions.

Compositions within the scope of this disclosure include compositions wherein the therapeutic agent is a system comprising a vector and an expression cassette in an amount effective to reduce or eliminate the growth and/or survival of a target cell such as a senescent cell, a cancer cell, a cell infected with an infectious agent or a cell that is associated with another disease or condition. Determination of optimal ranges of effective amounts of each component is within the skill of the art. The effective dose is a function of a number of factors, including the specific system, the presence of one or more additional therapeutic agent within the composition or given concurrently with the system, the frequency of treatment, and the patient's clinical status, age, health, and weight.

Compositions comprising a system may be administered parenterally. As used herein, the term “parenteral administration” refers to modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal, and intrasternal injection and infusion. Alternatively, or concurrently, administration may be orally.

Compositions comprising a system may, for example, be administered intravenously via an intravenous push or bolus. Alternatively, compositions comprising a system may be administered via an intravenous infusion.

Compositions include a therapeutically effective amount of a system, and a pharmaceutically acceptable carrier. As used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skimmed milk, glycerol, propylene, glycol, water, ethanol and the like. The composition, if desired, can also contain minor amounts of wetting or emulsifying agents, or pH buffering agents.

These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and the like. Such compositions will contain a therapeutically effective amount of the inhibitor, preferably in purified form, together with a suitable amount of carrier so as to provide the form for proper administration to the patient. The formulation should suit the mode of administration.

Compositions can be formulated in accordance with routine procedures as a pharmaceutical composition adapted for intravenous administration to a human. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The vectors disclosed herein can be formulated as neutral or salt forms. Pharmaceutically acceptable salts include those formed with anions such as those derived from hydrochloric, phosphoric, acetic, oxalic, tartaric acids, and the like, and those formed with cations such as those derived from sodium, potassium, ammonium, calcium, ferric hydroxides, isopropylamine, triethylamine, 2-ethylamino ethanol, histidine, procaine, and the like.

### Methods for Using Non-Native Pri-miRNA and miRNA Derived from Those Pri-miRNA

Within certain embodiments, the present disclosure provides methods for using the non-native pri-microRNAs (pri-miRNAs) described herein and for using the miRNA that derive from those pri-miRNA. Within certain aspects of those embodiments, disclosed herein are methods for regulating, promoting, normalizing, restoring, inhibiting, or modulating a desired cellular phenotype including, for example, differentiation, de-differentiation, proliferation, growth, cell death, contact inhibition by expressing one or more pri-miRNAs or one or more miRNAs identified through the screening of a pri-miRNA library according to the methodology disclosed herein.

Within other aspects of those embodiments, disclosed herein are methods for the treatment of a disease or condition that associated with the expression of one or more gene or the production of one or more protein, wherein one or more aspect of the disease or condition is reduced in severity following the expression of one or more pri-miRNAs or miRNAs identified through the screening of a pri-miRNA library according to the methodology disclosed herein.

Within further embodiments, the present disclosure provides methods for reducing, preventing, and/or eliminating the growth of a target cell, which methods comprise contacting a target cell with a vector system for the targeted production of one or more pri-miRNAs or miRNAs identified through the screening of a pri-miRNA library according to the methodology disclosed herein wherein the vector comprises: (a) a transcriptional promoter that is activated in response to one or more factors each of which factors is produced within a target cell and (b) a nucleic acid that is operably linked to and under regulatory control of the transcriptional promoter, wherein the nucleic acid encodes one or more pri-miRNAs or miRNAs identified through the screening of a pri-miRNA library according to the methodology disclosed herein and wherein production of the one or more pri-miRNAs or miRNAs reduces, prevents, and/or eliminates growth and/or survival of the target cell.

Within still further embodiments, the present disclosure provides methods for the treatment of a human that is afflicted with a disease or another condition, wherein the disease, or other condition is associated with a target cell within the human, the methods comprising administering to the human a vector for the production of one or more pri-miRNAs or miRNAs identified through the screening of a pri-miRNA library according to the methodology disclosed herein wherein the vector comprises an expression construct for the targeted production of one or more pri-miRNAs or miRNAs identified through the screening of a pri-miRNA library according to the methodology disclosed herein wherein the vector comprises: (a) a transcriptional promoter that is activated in response to one or more factors each of which factors is produced within a target cell and (b) one or more pri-miRNAs or miRNAs identified through the screening of a pri-miRNA library according to the methodology disclosed herein and wherein the nucleic acid is operably linked to and under regulatory control of the transcriptional promoter, wherein the one or more pri-miRNAs or miRNAs reduces, prevents, and/or eliminates growth and/or survival of the target cell thereby slowing, reversing, and/or eliminating the disease or condition in the human.

The amount of the one or more pri-miRNAs or miRNAs that will be effective in the treatment, inhibition, and/or prevention of cancer, infectious disease, or other disease or condition can be determined by standard clinical techniques. In vitro assays may optionally be employed to help identify optimal dosage ranges. The precise dose to be employed in the formulation will also depend on the route of administration, and the seriousness of the disease or disorder. Effective doses may be extrapolated from dose-response curves derived from in vitro or animal model test systems.

The systems or pharmaceutical compositions of the present disclosure can be tested in vitro, and then in vivo for the desired therapeutic or prophylactic activity, prior to use in humans. For example, in vitro assays to demonstrate the therapeutic or prophylactic utility of a compound or pharmaceutical composition include the effect of a system on a cell line or a patient tissue sample. The effect of the system or pharmaceutical composition thereof on the cell line and/or tissue sample can be determined utilizing techniques known to those of skill in the art including, but not limited to proliferation and apoptosis assays. In accordance with the present disclosure, in vitro assays that can be used to determine whether administration of a specific compound is indicated, include in vitro cell culture assays in which a patient tissue sample is grown in culture, and exposed to or otherwise administered a compound, and the effect of such compound upon the tissue sample is observed.

Methods of administration include, but are not limited to, intradermal, intramuscular, intraperitoneal, intravenous, subcutaneous, intranasal, epidural, and oral routes. The pri-miRNAs or miRNAs disclosed herein, or compositions thereof, may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the inhibitors or compositions into the central nervous system by any suitable route, including intraventricular and intrathecal injection. Intraventricular injection may be facilitated by an intraventricular catheter, for example, attached to a reservoir, such as an Ommaya reservoir. Pulmonary administration can also be employed, for example, by use of an inhaler or nebulizer, and formulation with an aerosolizing agent.

It may be desirable to administer the pri-miRNAs or miRNAs locally to the area in need of treatment; this may be achieved by, for example, local infusion during surgery, topical application, by injection, by means of a catheter, by means of a suppository, or by means of an implant, said implant being of a porous, non-porous, or gelatinous material, including membranes, such as sialastic membranes, or fibers.

The pri-miRNAs or miRNAs can be delivered in a controlled release system placed in proximity of the therapeutic target, thus requiring only a fraction of the systemic dose (see, e.g., Goodson, in Medical Applications of Controlled Release 2:115-138 (1984)).

Intravenous infusion of a compositions comprising a system may be continuous for a duration of at least about one day, or at least about three days, or at least about seven days, or at least about 14 days, or at least about 21 days, or at least about 28 days, or at least about 42 days, or at least about 56 days, or at least about 84 days, or at least about 112 days.

Continuous intravenous infusion of a composition comprising a system may be for a specified duration, followed by a rest period of another duration. For example, a continuous infusion duration may be from about 1 day, to about 7 days, to about 14 days, to about 21 days, to about 28 days, to about 42 days, to about 56 days, to about 84 days, or to about 112 days. The continuous infusion may then be followed by a rest period of from about 1 day, to about 2 days to about 3 days, to about 7 days, to about 14 days, or to about 28 days. Continuous infusion may then be repeated, as above, and followed by another rest period.

Regardless of the precise infusion protocol adopted, it will be understood that continuous infusion of a composition comprising one or more pri-miRNAs or miRNAs will continue until either desired efficacy is achieved or an unacceptable level of toxicity becomes evident.

It will be understood that, unless indicated to the contrary, terms intended to be “open” (e.g., the term “including” should be interpreted as “including but not limited to,” the term “having” should be interpreted as “having at least,” the term “includes” should be interpreted as “includes but is not limited to,” etc.). Phrases such as “at least one,” and “one or more,” and terms such as “a” or “an” include both the singular and the plural.

It will be further understood that where features or aspects of the disclosure are described in terms of Markush groups, the disclosure is also intended to be described in terms of any individual member or subgroup of members of the Markush group. Similarly, all ranges disclosed herein also encompass all possible sub-ranges and combinations of sub-ranges and that language such as “between,” “up to,” “at least,” “greater than,” “less than,” and the like include the number recited in the range and includes each individual member.

All references cited herein, whether supra or infra, including, but not limited to, patents, patent applications, and patent publications, whether U.S., PCT, or non-U.S. foreign, and all technical and/or scientific publications are hereby incorporated by reference in their entirety.

While various embodiments have been disclosed herein, other embodiments will be apparent to those skilled in the art. The various embodiments disclosed herein are for purposes of illustration and are not intended to be limiting, with the true scope and spirit being indicated by the claims.

The present disclosure will be further described with reference to the following non-limiting examples. The teaching of all patents, patent applications and all other publications cited herein are incorporated by reference in their entirety.

### EXAMPLES

### Example 1

### Pri-miRNA Scaffole Library Construction and Characterization

This Example demonstrates the construction and characterization of a representative pri-miRNA library comprising random pri-miRNA sequences based upon a miR-125a scaffold.

Sequences were uploaded to nupack.org and ViennaRNA for secondary structure (MFE) and binding analysis. These in silico simulations were used to finalize the design and ensure structural requirements for the actual construction of the inserts were met with minimal difference between wild-type miR-125a (miR-125a WT) and the modified version (randomiR-125a). The resulting oligos and primers were ordered from MWG|Operon (MWG) and assembled in-house.

Partial hairpins were extended using Klenow Fragment (exo-) (New England Biolabs; NEB), nicked by Nt.BspQI (NEB), gel purified on 4% agarose (Sigma-Aldrich; S-A) with 1× TBE buffer, extracted by Qiagen Gel Extraction Kit, and used in PCR (Phusion, NEB) with primers complementary to the flanking regions (MWG). The resulting products were amplified using additional primers (MWG) that encode AgeI and EcoRI restriction sites, as well as the flanking regions, and the PCR product was digested overnight with these AgeI and EcoRI (NEB) (10 hrs). The final product was gel-purified in a 4% agarose gel (S-A) with 1×TAE buffer and the desired band was isolated by gel extraction (Qiagen).

Oligonucleotides were analyzed with Operon's online oligo-analysis tool and melting temperatures of the primers, of the hairpins, and of their constituent domains are presented in Table 2.

Oligonucleotides are synthesized that are predicted to fold into a first “partial hairpin” comprising (a) a region 5′ to the random seed sequence on the miR: arm (5′ ss-flank, and lint of stem), (b) a randomized seed sequence, (c) remaining miR: sequence, (d) a terminal loop, and (e) a region of complementarity to the sequence 3′ to the random seed. A second partial hairpin is synthesized that is a reverse complement of the first partial hairpin, except that the 5′-most flanking region is replaced by the reverse complement of the 3′-flanking region in the complete pri-miRNA-like structure.

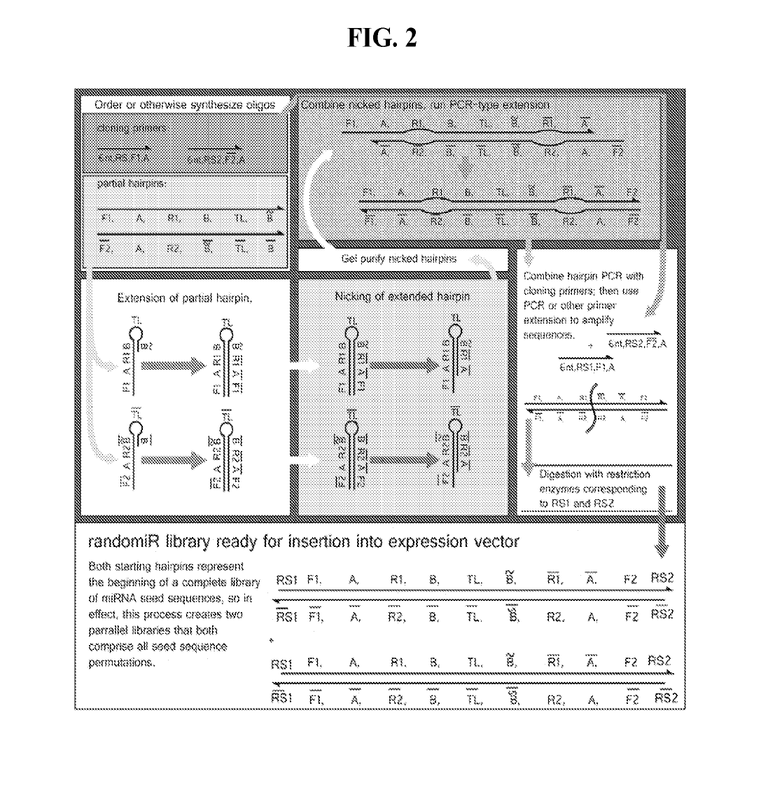

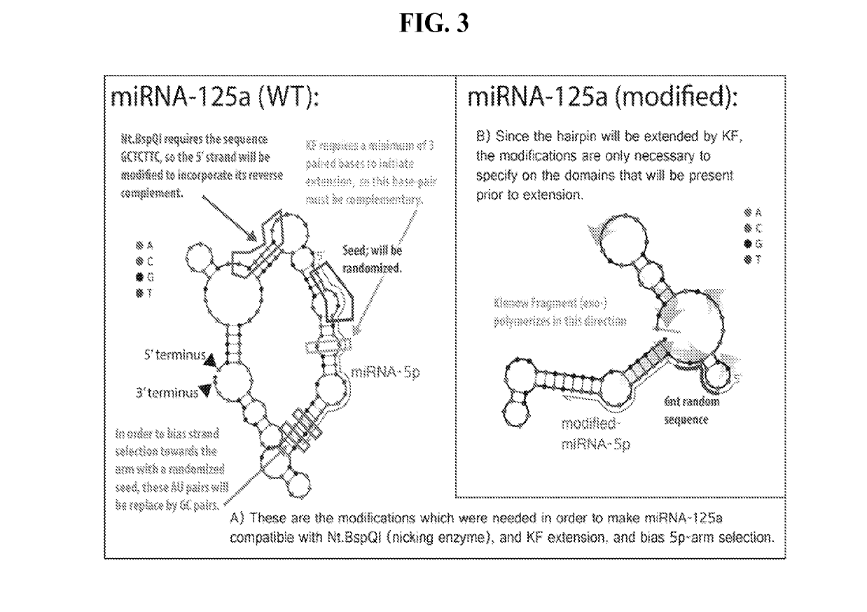

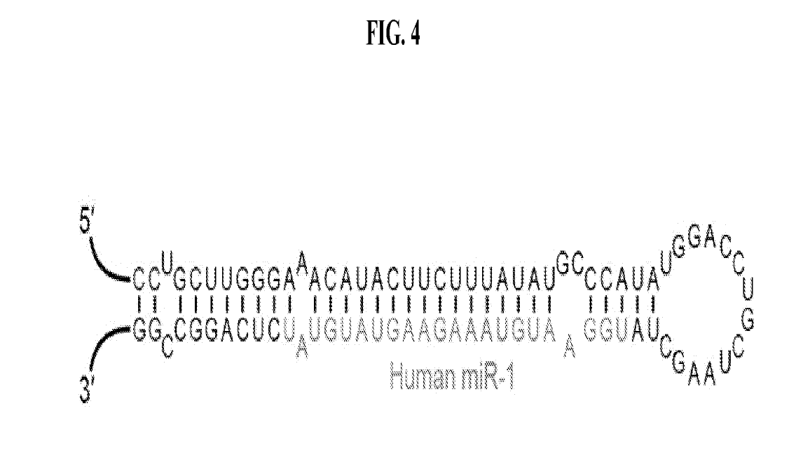

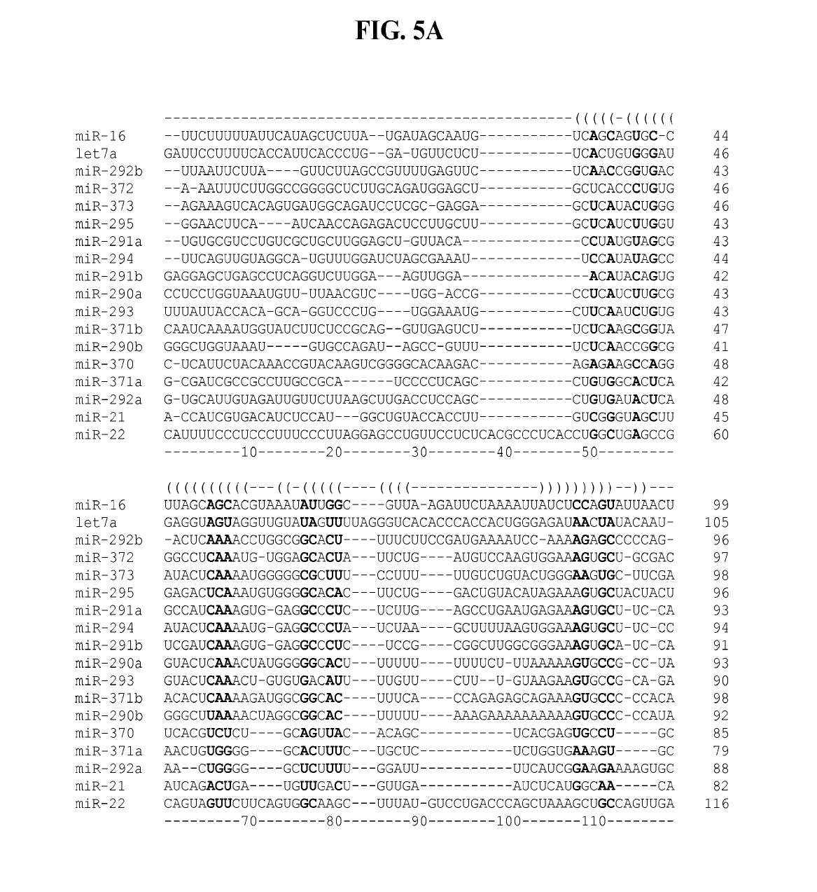

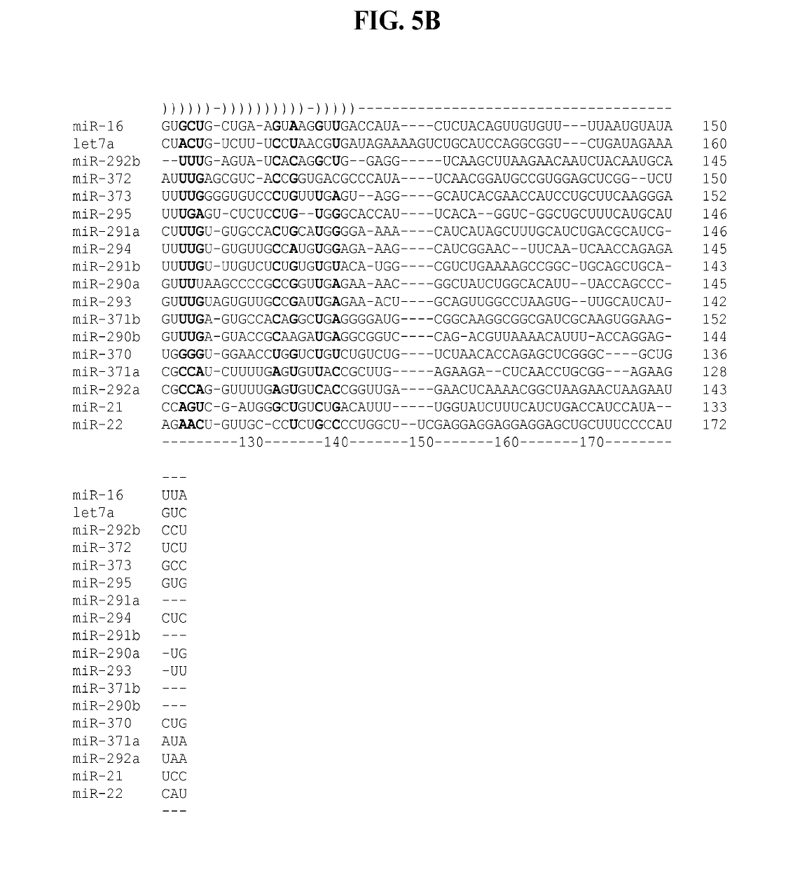

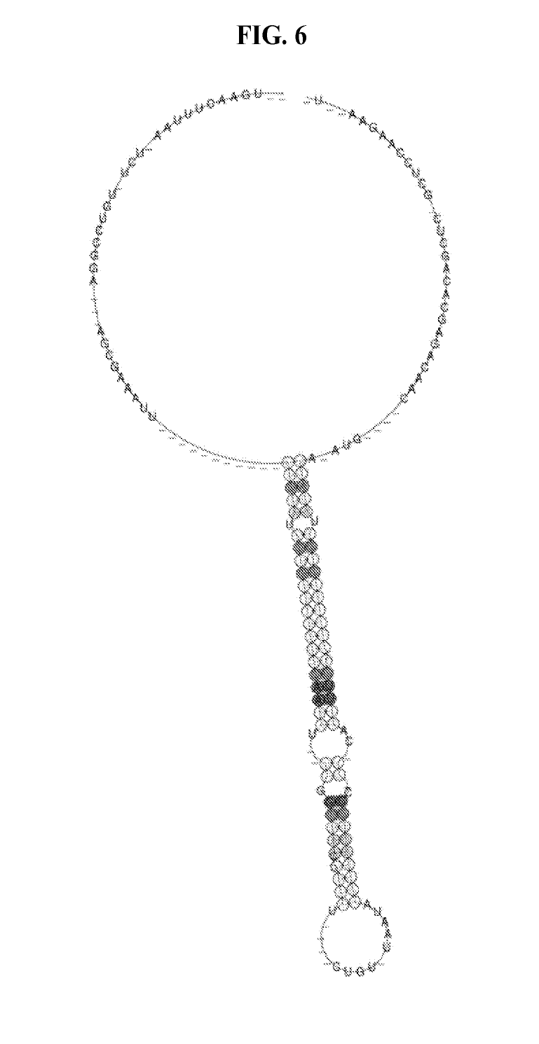

Using Klenow Fragment (exo-), or other suitable DNA polymerase, both hairpins are extended through the randomized region, stem, and flanking region to their 3′ ends, and the reactions are heat-killed. Next, a nicking enzyme is used to nick the DNA hairpins at the base of the stem, to remove the 3′ flanking regions that are complementary to the original 5′ flanking sequences, and the reaction is heat-killed. Nicked-and-extended hairpins are gel purified by a denaturing gel and the ˜0.135 nucleotide band is excised. Purified, nicked-and-extended-hairpins are combined, annealed, and used for PCR or other suitable DNA extension method to form complete hairpins having proper flanking sequences. Primers comprising 6 extra nucleotides, restriction sites, single-stranded flanking sequences, and 11 nucleotides of the stem are combined with complete hairpins, and the entire mixture is used in a PCR or other suitable DNA polymerization reaction. Following PCR and PCR purification, the resulting sample is restriction digested, gel purified, and the ˜145 nucleotide band excised. The length of the bands removed for gel purifications can be altered, as needed, for the construction of random pri-miRNAs based on different endogenous-miRNA backbones with different lengths. A diagram depicting this methodology described in this example is presented at FIG. 2.

It will be understood that the methods disclosed herein are designed to permit the construction of pri-miRNA libraries having complexities of at least 10⁴ and as high as 10⁹ wherein the sequences of the target binding regions are fully randomized within the 6 nucleotide seed sequence and at least partially randomized within the remaining 13-17 nucleotides constituting the 5′ and 3′ nucleotides flanking the 6 nucleotide seed sequence. Such pri-miRNA libraries are prepared in a manner such that they are not biased in favor of any pre-determined target mRNA sequence. Instead, the pri-miRNA libraries disclosed herein are designed to include fully-randomized seed sequences and at least partially-randomized flanking sequences such that individual pri-miRNAs having target binding specificity for any target mRNA are represented within the libraries and steps of clonal selection or biasing are eliminated. For example, when competent E. coli are used for transformation of plasmids containing a complete pri-miRNA library, the transformed E. coli are not plated for selection of individual colonies. Rather, the entirety of the transformed bacterial are grown in liquid culture without selection of individual clones.

Pri-miRNAs, including miR-125a, miR-124, miR-137, miR-145, miR-9, and let-7b or other naturally-occurring pri-miRNA, palindrome optimized variants of naturally-occurring pri-miRNA, or other suitable variants of naturally-occurring pri-miRNA can be modified according to the presently disclosed methods to generate pri-miRNA scaffolds that may be employed for the generation of pri-miRNA libraries according to the present disclosure. Regardless of the naturally-occurring pri-miRNA from which a pri-miRNA library is derived, its wild type seed sequence, and one or more nucleotides flanking its wild type seed sequence, may be modified by KF(exo-) extension throughout the mRNA target binding region adjacent to the Drosha cleavage site that is from 8 to 14 nucleotides 3′ to the first base of the 5′ palindromic sequence. Based on the miR-125a primary sequence and randomiR construction protocol, necessary changes were made to the miRNA sequence, as shown in FIG. 2. For the melting temperatures of the primers and each domain of the DNA sequences used in this embodiment of a randomiR library, and PCR temperature settings, see Table 1. Secondary structure predictions from nupack.org were used to verify theorized complex formation and to demonstrate the viability of this approach.

Custom primers are used to quantify amount of pri-miRNA that is transiently expressed in HeLa WT, and HeLa DKD, should be a decrease in expression from WT to DKD due to reduction in Drosha expression. Ct values are normalized to GAPDH. Sequencing methodologies are employed to demonstrate that sequences are random and to detect any change in bias from the plasmid library to actual expression in the form of mature miRNA.

Lentiviral infection of HeLa cells that are engineered to constitutively express GFP can be used to identify pri-miRNAs that can reduce the cellular levels of GFP by targeting mRNA encoding GFP for degradation via the RISC complex. In a preliminary study, a 9-12 nucleotide sequence including the canonical 6 nucleotide seed sequence is randomized prior to screening for the individual pri-miRNAs that are effective in facilitating the downregulation of GFP gene expression. hESC can then be infected a lentiviral vector encoding a panel of random pri-miRNA sequences and tested for enhanced neural differentiation by selecting pri-miRNAs that enhance GFP expression in Reelin-GFP, Nkx2.1-GFP, and GFAP-GFP hESC lines.

The generation of the pri-miRNA libraries disclosed herein may be advantageously employed to probe the elements of miRNA and mRNA sequences outside of the canonical seed region that determine the degree of translational repression effected by miRNA expression. Additionally, analysis of the expression of a pri-miRNA library amplified and mutated without selection may be used to study the effects of miRNA primary and secondary structure on miRNA maturation.

The theoretical validation of the exemplary miR-125a-based pri-miRNA library disclosed herein indicates the DNA strands will bind as needed for the protocol. The presently disclosed pri-miRNA libraries may be employed for the identification of individual miRNAs that promote cellular differentiation in vitro and, moreover, are well adapted to screening methodologies to permit the identification of multiple pri-miRNAs that act collectively to effect a desired cellular activity or to promote the differentiation of a stem cell into a committed cell type of a desired lineage, such as a neuronal cell. The presently disclosed libraries may also be employed to identify non-native miRNA seed sequences beyond those that are found in natural contexts and, in certain applications, are potent inducers of cellular differentiation.

What is claimed is:

1. A library of non-native pri-miRNAs, said library comprising non-native pri-miRNAs, wherein each non-native pri-miRNA comprises (i) one or more palindromic sequences for facilitating pri-miRNA hairpin formation; (ii) one or more Drosher/DGCR8 binding sequences; and (iii) one or more miRNA sequences comprising one or more non-native miRNA seed sequences and one or more non-native flanking sequences.

2. The library of non-native pri-miRNAs of claim 1 wherein said miRNA seed sequences or said flanking sequences are fully-randomized or partially-randomized.

3. The library of non-native pri-miRNAs of claim 2 wherein said miRNA seed sequences are fully-randomized.

4. The library of non-native pri-miRNAs of claim 2 wherein said miRNA seed sequences are fully-randomized.

5. The library of non-native pri-miRNAs of claim 1 wherein said library has a complexity of non-native pri-miRNAs of from 10⁴ distinct non-native pri-miRNAs to 10⁹ distinct non-native pri-miRNAs.

6. The library of non-native pri-miRNAs of claim 5 wherein said library has a complexity of non-native pri-miRNAs of from 10⁵ distinct non-native pri-miRNAs to 10⁸ distinct non-native pri-miRNAs.

7. The library of non-native pri-miRNAs of claim 6 wherein said library has a complexity of non-native pri-miRNAs of from 10⁶ distinct non-native pri-miRNAs to 10⁷ distinct non-native pri-miRNAs.

8. A method for the un-biased selection of a non-native pri-miRNA from a library of non-native pri-miRNAs, said method comprising the screening of a library of non-native pri-miRNAs for one or more non-native pri-miRNAs that can effect a phenotypic change upon a target cell.

9. The method of claim 8 wherein said target cell is a stem cell and wherein said phenotypic change is cellular differentiation to a partially or terminally differentiated cell.

10. The method of claim 9 wherein said partially or terminally differentiated cell is a neuronal cell.

11. The method of claim 8 wherein said target cell is a differentiated cell and wherein said phenotypic change is cellular de-differentiation to an undifferentiated cell.

12. The method of claim 11 wherein said undifferentiated cell is an induced pluripotent stem cell (iPSC).

13. The method of claim 8 wherein said target cell is a cell that is associated with a disease or condition and stem cell and wherein said phenotypic change is a modulation in cell growth, proliferation, or survival.

14. The method of claim 8 wherein two or more pri-miRNAs can, in combination, effect said phenotypic change upon said target cell.

15. A method for the un-biased identification of a non-native pri-miRNA that can effect a desired cellular function, activity, or phenotype, said method comprising identifying one or more non-native pri-miRNAs from a library of non-native pri-miRNAs wherein each of said one or more non-native pri-miRNAs (a) effects a desired cellular function, activity, or phenotype on a target cell, (b) forms a secondary structure comprising a double-stranded hairpin loop of from 17 base pairs to 25 base pairs, and (c) comprises a non-native seed sequence.

16. A method for preparing a non-native pri-miRNA library, said method comprising:
(a) identifying a naturally-occurring pri-miRNA comprising a palindromic sequence capable of adopting a secondary structure that includes hairpin structure having a Drosha cleavage site;
(b) obtaining a DNA encoding said naturally-occurring pri-miRNA;
(c) modifying said pri-miRNA encoding DNA by removing from 19 to 23 nucleotides that constitute the corresponding miRNA target recognition sequence and comprise a seed sequence of from 4 nucleotides to 8 nucleotides; and
(d) ligating into said pri-miRNA a DNA fragment of 19 to 23 nucleotides, wherein said DNA fragment comprises a seed sequence and one or more flanking sequence, wherein said seed sequence is fully randomized.

17. A method for preparing a non-native pri-miRNA library, said method comprising: engineering a non-naturally occurring DNA sequence comprising a 5′ region of from 19 to 23 nucleotides and a 3′ region that is complementary to said 5′ region, wherein said 5′ region is separated from said 3′ region by a sequence that is capable of adopting a secondary structure that includes a loop, and wherein said DNA sequence further comprises a fully randomized seed sequence.

18. A non-native pri-miRNA library constructed by the method of claim 16.

19. A non-native pri-miRNA library constructed by the method of claim 17.
